(19) United States
(12) Patent Application Publication
(10) Pub. No.: US 2010/0068229 A1
(43) Pub. Date: Mar. 18, 2010

(54) RECOMBINANT ANTIGENS OF HUMAN CYTOMEGALOVIRUS (HCMV)

(76) Inventors: Nicola Gargano, Rome (IT); Elisa Beghetto, Rome (IT); Andrea Spadoni, Rome (IT); Francesco De Paolis, Palestrina (IT)

Correspondence Address: Steinfl & Bruno, 301 N Lake Ave Ste 810, Pasadena, CA, 91101, US

(21) Appl. No.: 12/525,800

(22) PCT Filed: Feb. 5, 2008
(86) PCT No.: PCT/EP2008/000876

§ 371 Date: Aug. 4, 2009

(30) Foreign Application Priority Data
Feb. 7, 2007 (EP) 07101864.2

(51) Int. Cl. A61K 39/245; C07K 14/045; C07H 21/00; C12N 15/63; C12N 1/00; C12P 21/00; C12Q 1/70; A61K 31/7052; A61P 31/12

(52) U.S. Cl. 424/230.1; 530/402; 536/23.4; 435/320.1; 435/243; 435/69.3; 435/5; 514/44 R

(57) ABSTRACT

The invention described herein relates to a method for identifying the antigenic regions of HCMV proteins involved in the human B-cell response to HCMV infection, for combining such antigenic regions in the form of chimeric fusion products, and their use as diagnostic and immunogenic agents.

### FIELD OF THE INVENTION

The invention described herein relates to the technical field of the preparation of diagnostic means not applied directly to animals or human body.

The invention also furnishes compounds, methods for their preparation, methods for their use and compositions containing them, which are suitable for industrial application in the pharmaceutical and diagnostic fields, particularly for the detection and diagnosis of human cytomegalovirus infection, as well as for the treatment and prevention of said infection.

### BACKGROUND OF THE INVENTION

Early diagnosis is a priority and a highly desirable objective in all fields of therapy, particularly because it allows a considerable improvement in the patient's life and a concomitant saving for both health care systems and the patients. In the particular case of the invention described herein, early diagnosis is a very important issue in case of potential or existing cytomegalovirus infection in pregnant women, with particular concern for the health of the fetus, and in infected subjects, particularly those with impaired immunity.

Human cytomegalovirus (HCMV) is the vernacular name of human herpesvirus-5, a highly host-specific virus of the Herpesviridae family. Morphologically, HCMV is the largest virus in the family having a double-stranded DNA genome of 235 kbp encoding around 165 genes (Dolan et al., 2004, J. Gen. Virol. 85:1301-1312). HCMV, like all herpesviruses, undergoes latency and reactivation in the host. The virus is prevalent in the human population, with 50-90% reactivation in the host. The virus is prevalent in the human population, with 50-90% of adults becoming seropositive by the age of 50, depending upon both socioeconomic factors and geographic location (Gandhi and Khanna, 2004, Lancet Infect. 4:725-738). Humans are the only reservoir of HCMV. Primary infection with HCMV is generally asymptomatic and self-limiting in immunocompetent hosts, but results in a lifelong carrier state with periodic reactivation and shedding of virus from mucosal sites. In contrast, reactivation of latent virus in immunosuppressed adults may give rise to pneumonitis with very serious outcomes (Gandhi et al., 2003, Blood Rev. 17:259-264). Moreover, contracting primary infection during pregnancy may lead to miscarriages or to severe fetal disease in congenitally infected newborns (Revello and Gema, Clin. Microbiol. Rev. 2002, 15:680-715).

For an extensive overview of the problem of HCMV infection the reader is referred to the specific medical literature.

Diagnosis of HCMV infection is established by isolating the virus and/or viral products in the blood or body fluids, detecting specific nucleotide sequences with PCR, and detecting specific anti-HCMV antibodies produced by the host in response to the infection (Revello and Gema, Clin. Microbiol. Rev. 2002, 15:680-715).

Main challenges for clinicians are the diagnosis of primary HCMV infection in pregnant women and the diagnosis of congenital infection in their newborns. In both cases, to implement suitable therapies in good time and to avoid possible damage to the fetus and newborns, it is very important to establish if the viral infection has been contracted before or after conception in pregnant women. Moreover, it is essential determining when the vertical transmission from the mother to the fetus occurred.

Seroconversion during gestation and diagnosis of congenital infection in neonates are generally done by attempting to detect the presence of the various classes of anti-HCMV immunoglobulins (IgG, IgM, avidity of IgG), and to compare the immunological profiles of the mother versus her child. However, the available commercial assays do not provide enough sensitivity and specificity to allow a correct diagnosis of infection in all patients. Moreover, most of the currently available immunoassays use poorly defined viral antigens derived from HCMV-infected fibroblast cultures and may vary in their abilities to detect serum immunoglobulins. Finally, another problem in the context of HCMV serodiagnosis is the true classification of results due to the lack of a gold standard. Therefore, the availability of specific, sensitive and innovative diagnostic agents is desirable.

Numerous studies have found various different antigenic proteins of HCMV and the corresponding gene sequences have also been determined.

Among the most interesting proteins both for diagnostic and therapeutic purposes, in the form of vaccines, we should mention pp150, a viral large phosphorylated tegument protein, which has been shown to be most reliably detected by sera known to be antibody positive for HCMV. No sequence homology between this protein and the proteins of Epstein-Barr virus, varicella-zoster virus, and herpes simplex virus has been found, thus reducing risk of cross reactions with other viral proteins.

HCMV antigens have long been known and available, first of all as antigen mixtures obtained in various ways. For example, Greijer and colleagues (Greijer et al., J. Clin. Microbiol. 1999, 37:179-188) reported a specific combination of peptides derived from pp52 (UL44) and pp150 (UL32) for the specific and highly sensitive early detection of HCMV IgM, whereas a combination of peptides from pp150 (UL32), gB (UL55), and pp28 (UL99) was selected to give optimal and specific reactivity with HCMV IgG. On the basis of the results obtained with these peptide combinations, new, highly specific serodiagnostic assays were constructed. These assays had sensitivities of 98.9 and 96.4% for IgG and IgM, respectively, in comparison with the results obtained with the “gold standard,” the virion antigen-based ELISA.

From the results of this study it was concluded that specific combinations of highly defined synthetic peptides could replace complex HCMV virion extracts used in current serodiagnostics.

During the last ten years, several studies have reported the use of recombinant antigens for the serological diagnosis of HCMV infection and for therapeutic application (e.g. vaccines).

It should be stressed that all these antigens are obtained by means of molecular biology techniques that use the expression of proteins in bacterial and mammalian cells. None of the documents cited describe the technique of expression/exposure libraries of cDNA fragments deriving from the genome of HCMV in the lambda phage (phage display) to obtain fragments of antigens of the pathogen.

International patent application WO03/010198 discloses a vector of DNA expression and protein exposure as molecular fusion with the amino-terminal part of protein D (pD) of the lambda bacteriophage. This vector, called λKM4, differs from that used for expression experiments (see for example λgt11) in that the recombinant protein encoded by the foreign DNA fragment is expressed as fusion product with a protein of the bacteriophage itself and then exposed on the capsid. According to this approach, the phage exposes the protein fragment on the surface only if its open reading frame (ORF) coincides with pD. The size of the fragments of DNA cloned in the libraries is selected in order to represent a population of medium size ranging from 200 to 1000 nucleotide base pairs (bp), and, for statistical reasons, most of the out-of-frame sequences contain stop codons which do not allow translation and consequently exposure on the surface of the phage.

### SUMMARY OF THE INVENTION

It has now been found that the combination of the affinity selection and phage display techniques provides a method for the identification of specific antigen fragments of HCMV, in particular by applying affinity selection on phage display libraries of HCMV DNA fragments with a panel of sera from infected individuals. DNA fragments are obtained by enzymatic digestion of genomic DNA of the HCMV virus. With this method it proves possible to identify antigen fragments from very large libraries (i.e. expressing a large number of different sequences). The antigen fragments thus identified can be used for diagnostic and therapeutic purposes. Also, it has been found that the combination of antigenic regions of HCMV proteins, in the form of recombinant chimeric proteins, retains the antigenic properties of the individual antigen fragments and improves the performance of the diagnostic assays, in which they are used. The corresponding chimeric proteins thus produced can be used for diagnostic and therapeutic purposes.

Therefore, one object of the invention described herein is a method for the identification of antigen fragments of HCMV proteins, by applying affinity selection on phage display libraries of HCMV DNA fragments with a panel of sera from infected individuals.

The method provided by the present invention makes it possible to confirm the use of known HCMV antigens as diagnostic agents and also to identify in known antigens the epitopes that trigger an immune response in humans, and this portion is a further object of the present invention; but it also makes it possible to identify the antigenic function of HCMV proteins, for which such function was previously unknown; lastly, the method according to the present invention also provides new antigen fragments of HCMV gene products, that constitute yet another object of the present invention.

Another object of the present invention are antigen fragments isolated and characterised with the above-mentioned method, used as single recombinant proteins or combined as “antigen mixtures” or, by further genetic engineering, as chimeric antigens. The invention described herein also extends to the epitopes contained in said antigenic region.

The use of said recombinant proteins (the antigen fragments and the chimeric antigens obtained by combining two or more antigenic regions of the selected antigens) as diagnostic agents and the related diagnostic aids containing them, for example in the form of enzyme-linked immunoassays or kits or other supports, constitute a further object of the present invention.

The use of said antigen fragments and chimeric antigens as active agents for the preparation of formulations, and particularly in the form of vaccines, which are useful for the prevention and cure of the infection in humans, constitute a further object of the present invention.

Another object of the present invention are the gene sequences coding for the above-mentioned antigen fragments and chimeric antigens, their use as medicaments, particularly for the prevention and therapy of HCMV infection, e.g. as gene therapy. The present invention also extends to the gene sequences that hybridise with the sequences of the above-mentioned fragments under stringent hybridisation conditions.

These and other objects will be illustrated here below in detail, also by means of examples and figures.

### DETAILED DESCRIPTION OF THE INVENTION

The main object of the present invention is the provision of recombinant antigen fragments of HCMV gene products and the provision of recombinant chimeric antigens obtained through the fusion of different antigenic regions of HCMV proteins, and the use of the recombinant products thus obtained for developing selective diagnostic and therapeutic means.

The main advantages of the use of chimeric antigens of the present invention over the other types of antigens or antigen fragments known in the literature as reported above are the following and are evident when these antigens are used in diagnostic immunoassays using serum samples for detection of the infection:

With respect to the use of the entire HCMV antigen, prepared as lysed, whole-cell extract from infected cells, the recombinant chimeric antigens have the advantage of avoiding unspecific reactions due to the presence of other non-viral material and of providing a better reproducibility.

With respect to the use of single antigenic regions of HCMV antigens, the recombinant chimeric antigens show the advantage of improving the sensitivity of the assays in which they are used. In other words their use decreases or abolishes the occurrence of false negative responses.

With respect to the industrial applicability and production of a mixture or a collection of single antigenic regions, the advantage is that it is much easier to produce a single engineered construct containing three or more antigen regions rather than separately produce each single fragment and subsequently assemble them by an economic and reproducible method.

These and other advantages are shown in the Examples section.

The present invention comprises the construction of expression/exposure libraries of DNA fragments prepared from HCMV DNA, the selection of such libraries with sera of patients who have been infected by HCMV, the characterisation of the antigen fragments, and the use of said fragments for developing selective diagnostic and therapeutic means.

The method according to the present invention advantageously combines affinity selection and the power of phage display. What is meant by phage display, as understood by the person of ordinary skill in the art, is a strategy based on the selection of expression/exposure libraries in which small protein domains are exposed on the surface of bacteriophages containing the corresponding genetic information. A library of the phage-display type, constructed using DNA deriving from pathogenic organisms, makes it possible to exploit affinity selection, which is based on incubation of specific sera (reactive with the pathogen) with collections of bacteriophages that express portions of proteins of the pathogen on their capside and that contain the corresponding genetic information. The bacteriophages that specifically bind the antibodies present in the serum are easily recovered, remaining bound (by the antibodies themselves) to a solid support (e.g. magnetic beads); the non-specific ones, by contrast, are washed away. Direct screening, i.e. the analysis of the ability of single phage clones to bind the antibodies of a given serum, is carried out only at a later stage, when the complexity of the library (i.e. the different number of sequences) is substantially reduced, precisely as a result of the selection.

In particular the present invention covers a human cytomegalovirus (HCMV) antigen fragment consisting of an amino acid sequence selected from the group consisting of: SEQ ID NO: 2, SEQ ID NO: 4, SEQ ID NO: 6, SEQ ID NO: 8, SEQ ID NO: 10, SEQ ID NO: 12 and SEQ ID NO: 14, and mixtures thereof.

According to another embodiment the present invention covers a chimeric recombinant antigen containing the fusion of at least three different antigenic regions of HCMV proteins, wherein said antigenic regions are B-cell epitopes, which bind to HCMV-specific antibodies; preferably the HCMV-specific antibodies are extracted from sera of subjects who have been infected by HCMV.

Preferably in the chimeric antigens of the invention the three different antigenic regions are linked by a covalent bond or by a peptide linker; more preferably each of the three different antigenic regions consist of an amino acid sequence selected from the group of: SEQ ID NO: 2, SEQ ID NO: 4, SEQ ID NO: 6, SEQ ID NO: 8, SEQ ID NO: 10, SEQ ID NO: 12 and SEQ ID NO: 14.

According to a specific embodiment of the invention the chimeric antigen comprises the amino acid sequence of SEQ ID NO: 16 or the amino acid sequence of SEQ ID NO: 18.

The term “polypeptide” is ordinarily applied to a polypeptidic chain containing at least 4 contiguous amino acids, usually from 20 to 500 contiguous amino acids.

The term “epitope” referred to herein, relates to that part of an antigenic molecule that is recognized and bound by a T-cell receptor or by a B-cell receptor or by an antibody (i.e. a determinant on a large molecule against which an antibody can be produced and to which it will bind). The term as used herein is intended to include antigenic determinants of naturally occurring molecules or synthetic molecules that can mimic naturally occurring antigenic determinants.

Molecules which mimic the naturally occurring antigenic determinants may also be referred to as “mimotopes”, and these terms may be used interchangeably in reference to epitopes which are not formed by a contiguous segment of the primary sequence of an antigen.

According to the present invention an epitope is a polypeptidic chain from 9 to 40 amino acids long.

The term “antigen fragment” or “antigenic region” referred to herein, relates to a region of an antigenic molecule, which contains an epitope.

According to the present invention an antigen fragment is a polypeptidic chain from about 50 to about 500 amino acids long, preferably from 100 to 200.

The term “chimeric construct” or “fused construct” is herein used to refer to a polypeptide containing at least one of the amino acid sequences defined before. This polypeptide is thus encoded by a nucleic acid sequence created by joining the nucleic acid sequences coding for an isolated polypeptide of the invention and other antigen fragments containing immunodominant epitopes of HCMV gene products and also containing the essential nucleic acid sequences necessary for gene expression and replication in bacterial cells.

Preferentially, the other antigen fragments can be selected from a phage-display library of HCMV such as the antigen fragments described in the present invention and/or from antigenic regions of HCMV which are known in the literature.

The chimeric antigens of the present invention may be engineered using known methods. The fusions may be direct (the C-terminus of one amino acid sequence is linked to the N-terminal of the other through a simple covalent bond) or they may employ a flexible linker domain, such as the hinge region of human IgG, or polypeptide linkers consisting of small amino acids such as glycine, serine, threonine or alanine, at various lengths and combinations. For example the linker may be a polyglycine repeat interrupted by serine or threonine at a certain interval.

Preferably, the linker is composed by three glycine residues and two serine residues, giving the aminoacid sequence Ser-Gly-Gly-Gly-Ser (SGGGS linker).

Another object of the present invention is a nucleotide sequence coding for the chimeric antigen as defined above.

Preferably the nucleotide sequence of the invention is selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15 and SEQ ID NO: 16.

According to another aspect of the present invention, the nucleotide sequence comprises at least three different nucleotide sequences selected from the group consisting of: SEQ ID NO: 1, SEQ ID NO: 3, SEQ ID NO: 5, SEQ ID NO: 7, SEQ ID NO: 9, SEQ ID NO: 11 and SEQ ID NO: 13.

A nucleotide sequence that hybridizes with any sequence according to claims 8 to 10 under stringent hybridization conditions with any of the above-mentioned nucleotide sequences is also comprised in the scope of the present invention together with the chimeric recombinant antigen encoded by it.

Preferably the nucleotide sequence is a DNA sequence.

The recombinant antigens of the present invention may be prepared by cloning and expression in a prokaryotic or eukaryotic expression system, using the appropriate expression vectors. Any method known in the art can be employed.

For example the DNA molecules coding for the antigens of the invention are inserted into appropriately constructed expression vectors by techniques well known in the art (see Sambrook et al., 1989, Molecular Cloning: a laboratory manual, Cold Spring Harbor Laboratory Press, NY). Such vectors are another object of the present invention.

In order to be capable of expressing the desired protein (in this case the antigen fragments and chimeric antigens), an expression vector should comprise also specific nucleotide sequences containing transcriptional and translational regulatory information linked to the DNA coding the desired protein in such a way as to permit gene expression and production of the protein. First in order for the gene to be transcribed, it must be preceded by a promoter recognizable by RNA polymerase, to which the polymerase binds and thus initiates the transcription process. There are a variety of such promoters in use, which work with different efficiencies (strong and weak promoters).

For eukaryotic hosts, different transcriptional and translational regulatory sequences may be employed, depending on the nature of the host. They may be derived form viral sources, such as adenovirus, bovine papilloma virus, Simian virus or the like, where the regulatory signals are associated with a particular gene, which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. All these hosts are a further object of the present invention.

Nucleic acid molecules which encode the recombinant antigens of the invention may be ligated to a heterologous sequence so that the combined nucleic acid molecule encodes a fusion protein. Such combined nucleic acid molecules are included within the embodiments of the invention. For example, they may be joined to the DNA coding for a protein which allows purification of the recombinant antigen by only one step of affinity chromatography. This joined/fused protein may be for example Glutathione Sulpho Transferase (GST) to generate fusion products at the carboxy terminus of GST protein. The corresponding recombinant proteins expressed in the cytoplasm of transformed E. coli cells may be purified by affinity chromatography using a Glutathione-Sepharose resin. Alternatively, the joined/fused protein may be the polyhistidine tag (also known as His-tag) to generate fusion products either at the carboxy terminus or at the amino terminus of the recombinant protein. The corresponding recombinant product expressed in the cytoplasm of transformed E. coli cells may be purified by affinity chromatography using a nichel-chelate affinity-chromatography (for example the Ni-NTA resin from Qiagen, USA).

The DNA molecule comprising the nucleotide sequence coding for the antigen fragments of the invention is inserted into vector(s), having the operably linked transcriptional and translational regulatory signals, which is capable of replicating the desired gene sequences in the host cell. The cells which have been stably transformed by the introduced DNA can be selected by also introducing one or more markers which allow for selection of host cells which contain the expression vector. The marker may also provide for phototrophy to an auxotropic host, biocide resistance, e.g. antibiotics, or heavy metals such as copper, or the like. The selectable marker gene can either be directly linked to the DNA gene sequences to be expressed, or introduced into the same cell by co-transfection. Additional elements may also be needed for optimal synthesis of proteins of the invention.

Factors of importance in selecting a particular plasmid or viral vector include: the ease with which recipient cells, that contain the vector may be recognized and selected form those recipient cells which do not contain the vector; the number of copies of the vector which are desired in a particular host; and whether it is desirable to be able to “shuttle” the vector between host cells of different species.

Once the vector(s) or DNA sequence containing the construct(s) has been prepared for expression the DNA construct(s) mat be introduced into an appropriate host cell by any of a variety of suitable means: transformation, transfection, conjugation, protoplast fusion, electroporation, calcium phosphate-precipitation, direct microinjection, etc.

Host cells may be either prokaryotic or eukaryotic. Example of eukaryotic hosts are mammalian cells, such as human, monkey, mouse, and Chinese hamster ovary (CHO) cells. Expression in these host cells provides post-translational modifications to protein molecules, including correct folding or glycosylation at correct sites. Also yeast cells can carry out post-translational peptide modifications including glycosylation. A number of recombinant DNA strategies exist which utilize strong promoter sequences and high copy number of plasmids which can be utilized for production of the desired proteins in yeast.

Yeast recognizes leader sequences on cloned mammalian gene products and secretes peptides bearing leader sequences (i.e., pre-peptides). Example of prokaryotic hosts are bacteria, such as Escherichia coli.

After the introduction of the vector(s), the host cells are grown in a selective medium, which selects for the growth of vector-containing cells.

Expression of the cloned gene sequence(s) results in the production of the desired proteins.

Purification of the recombinant antigens is carried out by any one of the methods known for this purpose, i.e. any conventional procedure involving extraction, precipitation, chromatography, electrophoresis, or the like. A further purification procedure that may be used in preference for purifying the antigens of the invention is affinity chromatography using monoclonal antibodies which bind the target protein and which are produced and immobilized on a gel matrix contained within a column. Impure preparations containing the recombinant protein are passed through the column. The antigens will be bound to the column by the specific antibody while the impurities will pass through. After washing, the antigen is eluted from the gel by a change in pH or ionic strength.

Another aspect of the present invention is the process for the production of the recombinant antigen as described above, comprising culturing the host cell transformed with the vector containing the nucleotide sequence of the invention and isolating the desired product.

A further object of the present invention is a DNA molecule comprising the DNA sequence coding for the above fusion protein, as well as nucleotide sequences substantially the same.

“Nucleotide sequences substantially the same” includes all other nucleic acid sequences which, by virtue of the degeneracy of the genetic code, also code for the given amino acid sequence.

Another object of the present invention is a nucleotide sequence which hybridizes to the complement of the nucleotide sequence coding for the antigen fragments of the invention under highly stringent or moderately stringent conditions, as long as the antigen obtained maintains the same biological activity, i.e. ability to bind to antibodies against the parasite.

The term “hybridization” as used here refers to the association of two nucleic acid molecules with one another by hydrogen bonding. Typically, one molecule will be fixed to a solid support and the other will be free in solution.

Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethyleneglycol); and the stringency of the washing conditions following hybridization.

Stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid: Tm=81.5° C.+16.6 (LogM)+0.41 (% GC)−0.61 (% form)−500/L, where M is the molarity of monovalent cations, % GC is the percentage of G and C nucleotides in the DNA, % form is the percentage of formamide in the hybridization solution, and L is the length of the hybrid in base pairs. For each 1° C. that the Tm is reduced from that calculated for a 100% identity hybrid, the amount of mismatch permitted is increased by about 1%. Thus, if the Tm used for any given hybridization experiment at the specified salt and formamide concentrations is 10° C. below the Tm calculated for a 100% hybrid according to equation of Meinkoth, hybridization will occur even if there is up to about 10% mismatch.

As used herein, highly stringent conditions are those which are tolerant of up to about 15% sequence divergence, while moderately stringent conditions are those which are tolerant of up to about 20% sequence divergence. Without limitation, examples of highly stringent (12-15° C. below the calculated Tm of the hybrid) and moderately (15-20° C. below the calculated Tm of the hybrid) conditions use a wash solution of 2×SSC (standard saline citrate) and 0.5% SDS at the appropriate temperature below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5×Denhardt's reagent, 0.5% SDS, 100 μg/ml denatured, fragmented salmon sperm DNA at a temperature approximately 20° C. to 25° C. below the Tm. If mixed probes are used, it is preferable to use tetramethyl ammonium chloride (TMAC) instead of SSC (Ausubel, 1987-1998).

The term “nucleic acid molecule” also includes analogues of DNA and RNA, such as those containing modified backbones.

Another aspect of the invention is the use of chimeric antigens described above as medicaments. In particular, one of the main objects of the invention is use of chimeric antigens as active ingredients for the preparation of medicaments for the prevention or treatment of HCMV infection. The pharmaceutical compositions should preferably comprise a therapeutically effective amount of the chimeric antigens of the invention or the corresponding nucleotide sequence. Chimeric antigens of the invention may thus act as vaccines for the prevention or the treatment of HCMV infection. For the therapeutic application, where the preparation of medicaments or vaccines comes within the framework of general knowledge for further reference the reader is again referred to the patent literature cited in the present description.

According to yet another aspect of the present invention a polypeptide vaccine is provided. The two major types of polypeptide vaccine are: polypeptides mixed with adjuvant substances and polypeptides which are introduced together with an antigen presenting cell (APC) (Mayordomo et al. 1995, Nature Med. 1:1297).

The most common cells used for the latter type of vaccine bone marrow and peripheral blood derived dendritic cells, as these cells express co-stimulatory molecules that help activation of CTL. Presenting the polypeptide can be effected by loading the APC with a polynucleotide (e.g., DNA, RNA) encoding the polypeptide or loading the APC with the polypeptide itself.

In accordance with the first type of polypeptide vaccine, adjuvant substances that stimulate immunogenicity are mixed with the polypeptide in order to improve the immune response to the polypeptide. Immunological adjuvants have generally been divided into two basic types: aluminum salts and oil emulsions. Aluminum phosphate and hydroxide (alum) adjuvants induce elevated levels of antibody against antigens in alum-based vaccines above those obtained with the corresponding aqueous vaccine. Numerous alum-based vaccines, including methods of preparation thereof, were developed as, for example, disclosed in U.S. Pat. Nos. 5,747,653, 6,013,264, 6,306,404 and 6,372,223. However, aluminum compounds have not always enhanced the immunogenicity of vaccines.

The main components of the oil-based adjuvants are: oil, emulsifier and immunostimulant. The earliest types of emulsified oil-based adjuvants are Incomplete Freund's Adjuvant (IFA), consisting of an approximately 50:50 water-in-oil emulsion, and complete Freund's adjuvant (CFA), a similar preparation with inclusion of killed mycobacteria. The powerful antibody-stimulating effect of CFA has not been surpassed by any other adjuvant.

However, because of severe toxic reactions CFA can be used only for experimental purposes and not in human or veterinary vaccines. The use of IFA in humans has been limited to those clinical situations in which aqueous vaccines are relatively impotent and aluminum compounds have not provided enough adjuvant activity. Example of improved emulsions as vaccine adjuvants, by enhancing the immunogenicity of the antigen, include submicron emulsions as disclosed in U.S. Pat. No. 5,961,970 and solid fat nanoemulsions as disclosed in U.S. Pat. No. 5,716,637 for example.

The uptake of a polypeptide of the invention may be facilitated by a number of methods. For instance, a non-toxic derivative of the cholera toxin B subunit, or of the structurally related subunit B of the heal-labile enterotoxin of enterotoxic Escherichia coli may be added to the composition, as disclosed in U.S. Pat. No. 5,554,378.

A composition a polypeptide of the invention can be directly administered to an individual for immunizing the individual. Alternatively, in accordance with an embodiment of the invention, the polypeptides may be used to generate new antibodies with the attribute and activities of known monoclonal antibodies. Ex-vivo activation of T-cells by these polypeptides may also elicit the desired activity of immunostimulation. Thus, the composition can be used for inducing antibodies in an ex-vivo system and the induced antibodies can then be administered to an individual for treating the infection. The composition can also be used in an ex-vivo system to stimulate T-cells to be administered in a process of adoptive immunotherapy, as described in the art.

The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs.

The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination (s), reaction sensitivities, and tolerance/response to therapy. This amount can be determined by routine experimentation and is within the judgement of the clinician. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal ortranscutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared. Dosage treatment may be a single dose schedule or a multiple dose schedule.

The method of treating a mammal suffering from HCMV infection, comprising administering a therapeutically effective amount of the vaccine as described above represents one of the aspects of the present invention.

A further object of the present invention is the use of recombinant antigens as described above as active agents for the diagnosis of HCMV infections, in particular for the diagnosis of the time of infection.

Also the kits for the diagnosis of HCMV infection, containing at least one antigen fragment or a combination of antigen fragments or chimeric antigens according are part of the present invention. Such kits may be useful for the diagnosis of an acute and/or latent HCMV infection.

The recombinant antigens of the invention may be employed in virtually any assay format that employs a known antigen to detect antibodies. A common feature of all of these assays is that the antigen is contacted with the body component suspected of containing antibodies under conditions that permit the antigen to bind to any such antibody present in the component. Such conditions will typically be physiologic temperature, pH and ionic strength using an excess of antigen. The incubation of the antigen with the specimen is followed by detection of immune complexes comprised of the antigen.

Design of the immunoassays is subject to a great deal of variation, and many formats are known in the art. Protocols may, for example, use solid supports, or immunoprecipitation. Most assays involve the use of labeled antibody or polypeptide; the labels may be, for example, enzymatic, fluorescent, chemiluminescent, radioactive, or dye molecules. Assays which amplify the signals from the immune complex are also known; examples of which are assays which utilize biotin and avidin, and enzyme-labeled and mediated immunoassays, such as ELISA assays.

The immunoassay may be, without limitation, in a heterogenous or in a homogeneous format, and of a standard or competitive type. In a heterogeneous format, the polypeptide is typically bound to a solid matrix or support to facilitate separation of the sample from the polypeptide after incubation.

Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride (known as Immulon™), diazotized paper, nylon membranes, activated beads, and Protein A beads. For example, Dynatech Immulon™1 or Immulon™2 microtiter plates or 0.25 inch polystyrene beads (Precision Plastic Ball) can be used in the heterogeneous format. The solid support containing the antigenic polypeptides is typically washed after separating it from the test sample, and prior to detection of bound antibodies. Both standard and competitive formats are known in the art.

In a homogeneous format, the test sample is incubated with the combination of antigens in solution. For example, it may be under conditions that will precipitate any antigen-antibody complexes which are formed. Both standard and competitive formats for these assays are known in the art.

In a standard format, the amount of antibodies forming the antibody-antigen complex is directly monitored. This may be accomplished by determining whether labeled anti-xenogenic (e.g., anti-human) antibodies which recognize an epitope on anti-HCMV antibodies will bind due to complex formation. In a competitive format, the amount of antibodies in the sample is deduced by monitoring the competitive effect on the binding of a known amount of labeled antibody (or other competing ligand) in the complex.

Complexes formed comprising anti-HCMV antibody (or, in the case of competitive assays, the amount of competing antibody) are detected by any of a number of known techniques, depending on the format. For example, unlabeled antibodies in the complex may be detected using a conjugate of antixenogeneic IgG complexed with a label, (e.g., an enzyme label).

In an immunoprecipitation or agglutination assay format the reaction between the recombinant antigen and the antibody forms a network that precipitates from the solution or suspension and forms a visible layer or film of precipitate. If no anti-HCMV antibodies are present in the test specimen, no visible precipitate is formed.

The recombinant antigens of the invention will typically be packaged in the form of a kit for use in these immunoassays. The kit will normally contain in separate containers the combination of antigens (either already bound to a solid matrix or separate with reagents for binding them to the matrix), control antibody formulations (positive and/or negative), labeled antibody when the assay format requires same and signal generating reagents (e.g., enzyme substrate) if the label does not generate a signal directly. Instructions (e.g., written, tape, VCR, CD-ROM, etc.) for carrying out the assay usually will be included in the kit.

The diagnostic kits, which are the object of the present invention, are therefore known to the expert in the field. The invention will now be illustrated in greater detail by means of examples and figures.

### DESCRIPTION OF THE FIGURES

FIG. 1. Plasmid map of the bacterial expression vector pGEX-SN-Flag

FIG. 2. Schematic representation of the selected phage clones

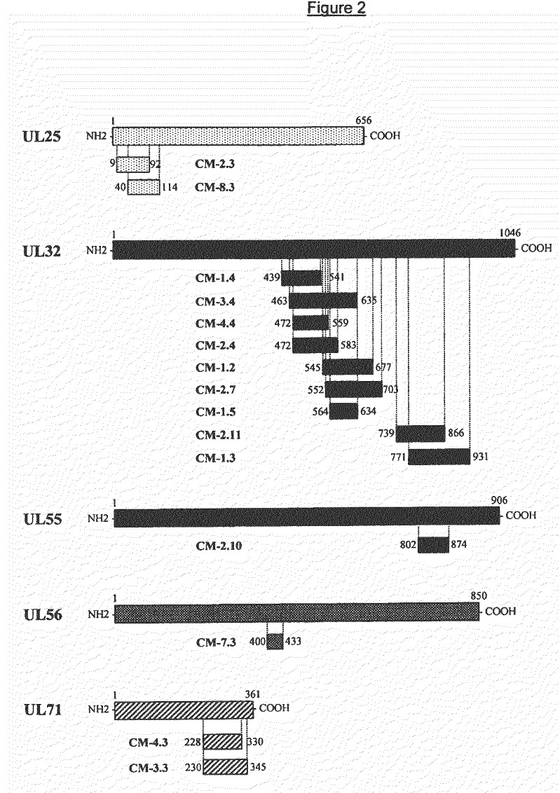

Alignment of the recombinant HCMV antigen fragments isolated from the phage display library with the sequence of the corresponding native proteins. The figure indicates the corresponding amino acids of each clone and their localization on HCMV protein sequences.

FIG. 3. Schematic representation of the recombinant chimeric antigens

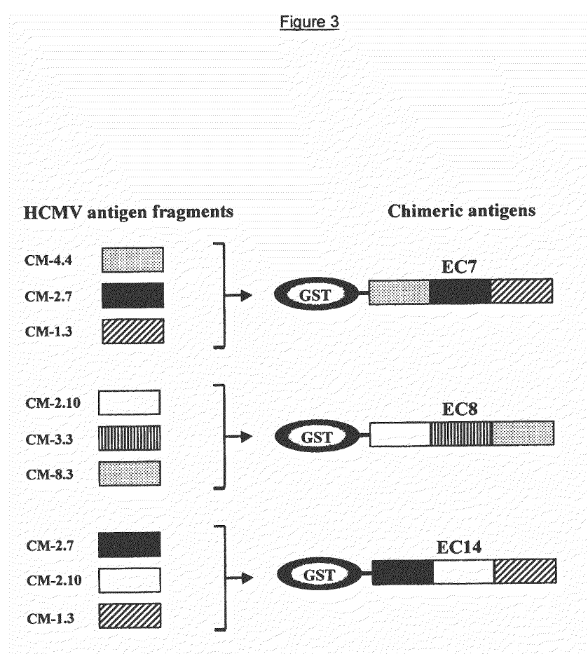

The DNA sequences of clones CM-4.4, CM-2.7, CM-1.3, CM-2.10, CM-3.3 and CM-8.3, encoding for protein fragments of HCMV gene products were used for the construction of GST-EC7-Flag, GST-EC8-Flag and GST-EC14 fusion proteins.

FIG. 4. Expression of recombinant antigens in E. coli cells

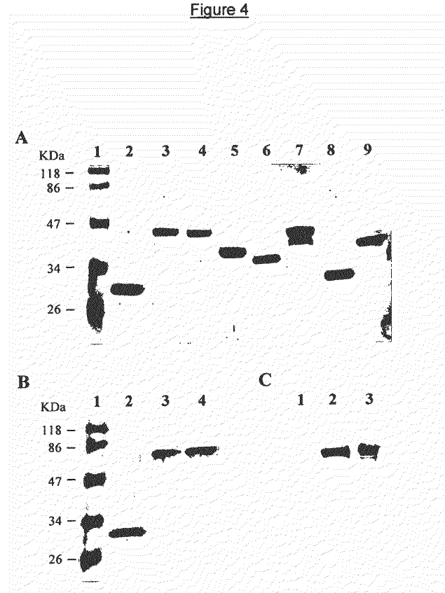

A—SDS-PAGE analysis of purified recombinant GST (lane 2), GST-CM1.3 (lane 3), GST-CM2.7 (lane 4), GST-CM4.4 (lane 5), GST-CM2.10 (lane 6), GST-CM3.3 (lane 7), GST-CM7.3 (lane 8) and GST-CM8.3 (lane 9) fusion proteins. The recombinant proteins were subjected to electrophoresis (0.002 mg/lane) on 12% acrylamide gel. KDa, molecular weight markers (lane 1)

B—SDS-PAGE analysis (12% acrylamide gel) of purified recombinant GST (lane 2) and chimeric antigens GST-EC7-Flag (lane 3) and GST-EC8-Flag (lane 4). KDa, molecular weight markers (lane 1)

C—Western Blot analysis of purified recombinant GST (lane 1), and chimeric antigens GST-EC7-Flag (lane 2) and GST-EC8-Flag (lane 3) employing an anti-Flag horse-radish peroxidase-conjugated monoclonal antibody.

### EXAMPLES

### Construction of the Lambda-Display HCMV DNA Library

Genomic DNA from HCMV (AD169 strain) was commercially available (Advanced Biotechnology, MD, USA). 10 μg of total DNA were fragmented randomly using 0.5 ng of the endonuclease DNasel (Sigma-Aldrich, USA). The mixture of DNA and DNasel was incubated for 20 minutes at 15° C. and the DNA fragments were purified by means of the “QIAquick PCR Purification Kit” (Qiagen, CA, USA), following the manufacturer's instructions. The ends of the DNA fragments were “flattened” by incubating the DNA with the enzyme T4 DNA polymerase (New England Biolabs, MA, USA) for 60 minutes at 15° C. The fragments were then purified by means of extraction in phenol/chloroform and subsequent precipitation in ethanol. The resulting DNA were ligated with a 20-fold molar excess of “synthetic adaptors” using the enzyme T4 DNA ligase for the purposes of adding the restriction sites SpeI and NotI to the ends of the fragments. Six adaptors were used, accordingly to the procedure previously described by Beghetto et al. (Beghetto et al., Int. J. Parasitol. 2003, 33:163-173). The excess of unligated adaptors was removed from the ligation mixture by electrophoresis on 2% agarose gel and the DNA fragments with molecular weights ranging from 200 by to 1000 base pairs (bp) were excised from the gel and purified by means of the “Qiaquick gel extraction kit” (Qiagen, CA, USA) following the manufacturer's instructions. The vector λKM4 was digested with SpeI/NotI and then ligated with DNA fragments for the construction of the library 6 ligation mixtures were performed, each containing 0.4 μg of vector and approximately 7 ng of insert. After overnight incubation at 4° C. with the enzyme T4 DNA ligase the ligation mixtures were packaged in vitro with the “Gigapack gold” (Stratagene, USA) and plated for infection of BB4 cells (bacterial cells of E. coli strain BB4; Sambrook et al., 1989, Molecular Cloning: a laboratory manual, Cold Spring Harbor Laboratory Press, NY). After overnight incubation at 37° C. the phage was eluted from the plates with SM buffer (Sambrook et al., 1989, Molecular Cloning: a laboratory manual, Cold Spring Harbor Laboratory Press, NY), purified, concentrated and stored at −80° C. in SM buffer containing 7% dimethylsulphoxide. The complexity of the library calculated as the number of total independent clones with inserts was 2×10⁶ clones.

Affinity Selection of the HCMV Display Library with Human Sera

Magnetic beads coated with Protein G (Dynabeads Protein-G, Dynal, Norway) were incubated with 10 μl of human serum for 30 minutes at room temperature. The beads were then incubated for 1 hour at 37° C. with blocking solution consisting of: 5% skimmed milk powder in PBS, 0.05% Tween 20, and 10 mM MgSO₄. Approximately 10¹⁰ phage particles of the library were added to the beads and diluted in 1 ml of blocking solution for a further 4-hour incubation at room temperature with weak stirring. The beads were washed 10 times with 1 ml of washing solution (PBS, 1% TritonX100, 10 mM MgSO₄). The bound bacteriophages were amplified for infection of BB4 cells added directly to the beads (1.2 ml per selection) and subsequent 30-minute incubation at room temperature. NZY-Top Agar (Sambrook et al., 1989, Molecular Cloning: a laboratory manual, Cold Spring Harbor Laboratory Press, NY) were added to the mixture of beads and cells were immediately poured onto NZY plates. The plates were incubated for 12-16 hours at 37° C. Next day the phages were collected from the plates by means of the addition of 15 ml of SM buffer per plate and stirring for 4 hours at room temperature. The phages were purified by precipitation in PEG/NaCl (20% polyethylene glycol, NaCl 1M) and finally suspended in 5 ml of SM and stored at +4° C.

### Phage-ELISA

Multi-well plates (Maxisorb, Nunc, Denmark) were coated overnight at 4° C. with anti-lambda polyclonal antibodies (0.7 μg/ml in NaHCO₃ 50 mM, pH 9.6). After eliminating the coating solution, the plates were incubated for 1 hour with blocking solution (5% skimmed milk powder in PBS, 0.05% Tween-20) and then washed twice with washing buffer (PBS, 0.05% Tween-20). A mixture of 100 μl of blocking solution containing phage lysate was added to each well and incubated for 60 minutes at 37° C. 1 μl of human serum was incubated for 30 minutes at room temperature with 10⁹ wild-type phage particles, 1 μl of rabbit serum, 1 μl of bacterial extract of BB4 cells, 1 μl of foetal bovine serum in 100 μl of blocking solution. The plates were washed 5 times after incubation with the phage lysate and then incubated with the serum solution for 60 minutes at 37° C. The plates were then washed 5 times and then incubated 30 minutes with blocking solution containing anti-human IgG horseradish peroxidase-conjugated antibodies (Sigma-Aldrich, USA). The plates were washed 5 times and the enzyme activity was measured with 100 μl of TMB liquid substrate (Sigma-Aldrich, USA). After 15 minutes' development, the reaction was stopped by adding 25 μl of H₂SO₄ 2M. Lastly, the plates were analysed using an automatic ELISA reader (Multiskan, Labsystem, Finland) and the results were expressed as OD=OD₄₅₀ₙₘ−OD₆₂₀ₙₘ. The ELISA data were assessed as mean values of two independent assays.

### Immunoscreening

Phage plaques were transferred from the bacterial medium to nitrocellulose filters (Schleicher & Schuell, Germany) by means of incubation at room temperature for 60 minutes. The filters were blocked for 60 minutes at room temperature in blocking solution (5% skimmed milk powder in PBS, 0.05% Tween-20). 40 μl of human serum were preincubated with 40 μl of bacterial extract of BB4 cells, 10⁹ wild-type lambda phage particles in 4 ml of blocking solution. After eliminating the blocking solution, the filters were incubated with the serum for 3 hours at room temperature under stirring. The filters were then washed 5 times with washing buffer (PBS, 0.05% Tween-20) and then incubated for 60 minutes at room temperature with anti-human IgG alkaline phosphatise-conjugated antibodies (Sigma-Aldrich, USA) in blocking solution.

After washing the filters 5 times, 5 ml of development solution (substrates BCIP and NBT, Sigma-Aldrich, USA) were added and the development was interrupted by washing the filters in water. Phage clones that proved positive were isolated from the respective phage plaques and then amplified for subsequent characterisation (Sambrook et al., 1989, Molecular Cloning: a laboratory manual, Cold Spring Harbor Laboratory Press, NY).

### Characterisation of Positive Clones

The DNA inserts of the selected phage were subsequently sequenced and compared with various databases of sequences currently available (Non-Redundant Genbank CDS, Non-Redundant Database of Genbank Est Division, Non-Redundant Genbank+EMBL+DDBJ+PDB Sequences).

The sequences obtained can be classified in three groups:

sequences that code for fragments of known HCMV antigens;

sequences that code for fragments of known HCMV proteins which, however, are not known to be involved in the human antibody response;

sequences that code for fragments of unknown proteins (e.g. ORF);

The following Table 1 gives, by way of examples, the sequences of some of the clones selected:

TABLE 1

| | | Identi- | Classi- |
|---|---|---|---|
| Name | Sequence | fication | fication |
| CM-2.10 | ACCAAAGACACGTCGTTACAGGCT | UL55 | Glycopro- |
| (SEQ ID | CCGCCTTCCTACGAGGAAAGTGTT | (frag- | tein-B, |
| 1) | TATAATTCTGGTCGCAAAGGACCG | ment) | envelope |
| | GGACCACCGTCGTCTGATGCATCC | | protein |
| | ACGGCGGCTCCGCCTTACACCAAC | | |
| | GAGCAGGCTTACCAGATGCTTCTG | | |
| | GCCCTGGCCCGTCTGGACGCAGAG | | |
| | CAGCGAGCGCAGCAAAACGGTACA | | |
| | GATTCTTTGGACGGACAGACTGGC | | |
| | ACGCAT | | |
| CM-3.3 | GCTCACATTAACACCGTCTCCTGT | UL71 | Hypothet- |
| (SEQ ID | CCTACCGTTATGAGGTTCGACCAG | (frag- | ical |
| 3) | CGGCTGCTGGAAGAGGGCGACGAG | ment) | protein |
| | GAGGATGAAGTGACCGTGATGTCG | | |
| | CCGTCACCCGAGCCCGTGCAACAG | | |
| | CAGCCGCCGGTCGAGCCCGTGCAG | | |
| | CAGCAGCCCCAGGGACGCGGGTCT | | |
| | CACCGTCGGCGCTACAAGGAGTCG | | |
| | GCGCCGCAAGAGACGCTGCCTACG | | |
| | AATCACGAACGCGAGATTTTGGAT | | |
| | CTCATGCGACACAGCCCCGACGTG | | |
| | CCTCGGGAGGCGGTGATGTCACCG | | |
| | ACCATGGTCACCATACCTCCTCCC | | |
| | CAGATACCCTTTGTGGGTTCCGCG | | |
| | CGTGAACTT | | |
| CM-83 | TCACGTCGCTCTGGCGAACCCTCG | UL25 | Tegument |
| (SEQ ID | ACGGTGATTTATATCCCCTCGAGC | (frag- | protein |
| 5) | AACGAGGACACGCCGGCGGATGAG | ment) | |
| | GAGGCGGAGGACAGCGTTTTCACG | | |
| | AGCACGCGGGCGCGCAGCGCCACG | | |
| | GAAGATCTGGATCGCATGGAGGCC | | |
| | GGTTTGTCGCCCTACAGCGTCTCC | | |
| | TCGGACGCTCCGTCGTCCTTCGAG | | |
| | CTCGTGCGCGAGACCGGCGGCACC | | |
| | GGCGCCGCCAAGAAACCGAGCGAA | | |
| | AAGAAACGATCGTTT | | |
| CM-7.3 | CTTATTCTCGAGGAGATTCGACGT | UL56 | viral DNA |
| (SEQ ID | CCGCTGCCAGATGGCACGGGGGGC | (frag- | binding |
| 7) | GACGGCCCCGAGGGCGAGGCTATT | ment) | protein |
| | CACCTGCGTGGACGGGAGGCGCAT | | |
| CM-1.3 | ACGAGCCAGAAACCGGTGCTGGGC | UL32 | Tegument |
| (SEQ ID | AAGCGAGTCGCGACGCCGCACGCG | (frag- | phospho- |
| 9) | TCCGCCCGAGCGCAGACGGTGACG | ment) | protein |
| | TCGACGCCGGTTCAGGGAAGGCTA | | (pp150) |
| | GAGAAACAGGTGTCGGGCACGCCG | | |
| | TCGACGGTACCCGCCACGCTGTTG | | |
| | CAACCTCAACCGGCTTCGTCTAAA | | |
| | ACGACGTCATCAAGGAACGTGACT | | |
| | TCTGGCGCGGGAACCTCTTCCGCT | | |
| | TCTTCGGCTCGACAGCCGTCAGCC | | |
| | TCGGCGTCCGTTTTGTCGCCCACG | | |
| | GAGGATGATGTCGTGTCCCCCGCC | | |
| | ACATCGCCGCTGTCCATGCTTTCG | | |
| | TCAGCCTCTCCGTCCCCGGCCAAG | | |
| | AGTGCCCCCCCGTCTCCGGTGAAA | | |
| | GGCCGGGGCAGCCGCGTCGGTGTT | | |
| | CCTTCCTTGAAACCTACTTTGGGC | | |
| | GGCAAGGCGGTGGTAGGTCGACCG | | |
| | CCCTCGGTCCCCGTGAGCGGTAGC | | |
| | GCGCCGGGTCGCCTGTCCGGCAGC | | |
| CM-2.7 | CTGGTGGACATCACGGATACCGAG | UL32 | Tegument |
| (SEQ ID | ACGAGCGCCAAACCGCCCGTCACC | (frag- | phospho- |
| 11) | ACCGCGTACAAGTTCGAGCAACCG | ment) | protein |
| | ACGTTGACGTTCGGCGCCGGAGTT | | (pp150) |
| | AACGTTCCTGCTGGCGCCGGCGCT | | |
| | GCCATCCTCACGCCGACGCCTGTC | | |
| | AATCCTTCCACGGCCCCCGCTCCG | | |
| | GCCCCGACACCTACCTTCGCGGGT | | |
| | ACCCAAACCCCGGTCAACGGTAAC | | |
| | TCGCCCTGGGCTCCGACGGCGCCG | | |
| | TTGCCCGGGGATATGAACCCCGCC | | |
| | AACTGGCCGCGCGAACGCGCGTGG | | |
| | GCCCTCAAGAATCCTCACCTGGCT | | |
| | TACAATCCCTTCAGGATGCCTACG | | |
| | ACTTCCACGGCTTCTCAAAACACC | | |
| | GTGTCCACCACCCCTCGGAGGCCG | | |
| | TCGACTCCACGCGCCGCGGTGACA | | |
| | CAAACAGCGTCTCGGGACGCCGCT | | |
| | GATGAGGTTTGGGCTTTAAGGGAC | | |
| | CTT | | |
| CM-4.4 | GGCAGTCAGAAACCGACCAGCGGT | UL32 | Tegument |
| (SEQ ID | CCCTTGAACATCCCGCAACAACAA | (frag- | phospho- |
| 13) | CAGCGTCACGCGGCTTTCAGTCTC | ment) | protein |
| | GTCTCCCCGCAGGTGACCAAGGCC | | (pp150) |
| | AGCCCGGGAAGGGTCCGTCGGGAC | | |
| | AGCGCGTGGGACGTGAGGCCGCTC | | |
| | ACGGAGACCAGAGGGGATCTTTTC | | |
| | TCGGGCGACGAGGATTCCGACAGC | | |
| | TCGGATGGCTATCCCCCCAACCGT | | |
| | CAAGATCCGCGTTTCACCGACACG | | |
| | CTGGTGGACATCACGGATACCGAG | | |
| | ATT | | |

The sequence CM-2.10 constitutes a DNA fragment of the HCMV genome, classified as UL55 and encoding for a fragment of the envelope glycoprotein gB (Pereira et al., Virol., 1984 139:73-86) that has never been identified as an “antigen fragment” recognized by the human antibody response. Said clone has the amino acid sequence TKDTSLQAPPSYEESVYNSGRKGPGPPSSDASTAAPPYTNEQAYQMLLALARLDA EQRAQQNGTDSLDGQTGTH (SEQ ID 2) and its use as a fragment containing an epitope is covered by the present invention.

The sequence CM-3.3 constitutes a DNA fragment of the HCMV genome, classified as UL71 (Davison et al., J. Gen. Virol. 2003, 84:17-28) and encoding for a polypeptide that has never been identified as an “antigen” recognized by the human humoral response. Said clone has the amino acid sequence AHINTVSCPTVMRFDQRLLEEGDEEDEVTVMSPSPEPVQQQPPVEPVQQQPQGR GSHRRRYKESAPQETLPTNHEREILDLMRHSPDVPREAVMSPTMVTIPPPQIPFVG SAREL (SEQ ID 4) and its use as a fragment containing an epitope is covered by the present invention.

The sequence CM-8.3 constitutes a DNA fragment of the HCMV genome, classified as UL25 and encoding for a fragment of a tegument antigen (Lazzarotto et al., J. Gen. Virol. 2001, 82:335-338) that has never been identified as an “antigen fragment” recognized by the human antibody response. Said clone has the amino acid sequence SRRSGEPSTVIYIPSSNEDTPADEEAEDSVFTSTRARSATEDLDRMEAGLSPYSVSS DAPSSFELVRETGGTGAAKKPSEKKRSF (SEQ ID 6) and its use as a fragment containing an epitope is covered by the present invention.

The sequence CM-7.3 constitutes a DNA fragment of the HCMV genome, classified as UL56 (Krosky et al., J. Virol. 1998, 72:4721-4728) and encoding for a polypeptide that has never been identified as an “antigen fragment” recognized by the human humoral response. Said clone has the amino acid sequence LILEEIRRPLPDGTGGDGPEGEAIHLRGREAH (SEQ ID 8) and its use as a fragment containing an epitope is covered by the present invention.

The sequences CM-1.3, CM-2.7 and CM-4.4 constitute DNA fragments of the HCMV genome, classified as UL32 and encoding for fragments of the large structural tegument phosphoprotein pp150 (Jahn et al., J. Virol. 1987, 61:1358-1367) that have never been identified as “antigen fragments” recognized by the human antibody response. Said clones have the respective amino acid sequences TSQKPVLGKRVATPHASARAQTVTSTPVQGRLEKQVSGTPSTVPATLLQPQPASSK TTSSRNVTSGAGTSSASSARQPSASASVLSPTEDDWSPATSPLSMLSSASPSPAK SAPPSPVKGRGSRVGVPSLKPTLGGKAWGRPPSVPVSGSAPGRLSGS (SEQ ID 10), LVDITDTETSAKPPVTTAYKFEQPTLT FGAGVNVPAGAGAAILTPTPVNPSTAPAPAPTPTFAGTQTPVNGNSPWAPTAPLPG DMNPANWPRERAWALKNPHLAYNPFRMPTTSTASQNWSTTPRRPSTPRAAVTQT ASRDAADEVWALRDL (SEQ ID 12), and GSQKPTSGPLNIPQQ QQRHAAFSLVSPQVTKASPGRVRRDSAWDVRPLTETRGDLFSGDEDSDSSDGYP PNRQDPRFTDTLVDITDTEI (SEQ ID 14), and their use as fragments containing epitopes are covered by the present invention.

### Construction of Chimeric Antigens

EC7 protein product is a chimeric molecule containing the DNA sequences of clones CM-1.3, CM-2.7 and CM-4.4.

SEQ ID 14 was used as template for DNA amplification of clone CM-4.4 by using oligonucleotides K749 (5′-GGACTAGTGGCAGTCAGAAACCGACCAG-3′) and K751 (5′-GGACTAGTGGCAGTCAGAAACCGACCAG-3′). The oligonucleotide K751 contains a sequence encoding for the linker SGGGS, which joins the sequences CM-4.4 and CM-2.7. PCR condition was 30″ at 94° C., 30″ at 52° C. and 30″ at 72° C. for 25 cycles.

SEQ ID 12 was used as template for DNA of clone CM-2.7 by using oligonucleotides K750 (5′-TCTGGTGGCGGTAGCCTGGTGGACATCACGG ATAC-3′) and K753 (5′-CGTGCTACCGCCACCAGAAAGGTCCCTTAAAGCCC AAAC-3′). The oligonucleotide K753 contains a sequence encoding for the linker SGGGS, which joins the sequences CM-2.7 and CM-1.3. PCR condition was 30″ at 94° C., 30″ at 52° C. and 30″ at 72° C. for 25 cycles.

SEQ ID 10 was used as template for DNA amplification of clone CM-1.3 by using oligonucleotides K752 (5′-TCTGGTGGCGGTAGCACGAGCCAGAAACCGGTGCTG-3′) and K754 (5′-CCGCGGCCGCTGGACACGACATCATCCTCC-3′). PCR condition was 30″ 94° C., 30″ at 52° C. and 30″ at 72° C. for 25 cycles.

The PCR products were purified by means of the “Qiagen Purification Kit” (Qiagen, CA, USA). 50 ng of DNA amplification products of SEQ ID 14 and SEQ ID 12 were mixed together and used as templates in PCR reaction by using oligonucleotides K749 and K753. PCR condition was 30″ at 94° C., 30″ at 52° C. and 90″ at 72° C. for 30 cycles. 50 ng of the resulting DNA amplification was purified with “Qiagen Purification Kit” (Qiagen, CA, USA) and then mixed with 50 ng of DNA amplification product of SEQ ID 10. Finally, the DNA mixture was used as template for DNA amplification by using K749 and K754, following PCR condition of 30″ at 94° C., 30″ at 52° C. and 120″ at 72° C. for 30 cycles.

EC8 protein product is a chimeric molecule containing the DNA sequences CM-2.10, CM-3.3 and CM-8.3.

SEQ ID 4 was used as template for DNA amplification of clone CM-3.3 by using oligonucleotides K761 (5′-GGACTAGTGCTCACATTAACACCGTCTC-3′) and K762 (5′-GTGAGCTACCGCCACCAGAAAGTTCACGCGCGGAAC-3′).

The oligonucleotide K762 contains a sequence encoding for the linker SGGGS, which joins the sequences CM-3.3 and CM-8.3. The PCR protocol was 30″ at 94° C., 30″ at 52° C. and 30″ at 72° C. for 25 cycles.

SEQ ID 6 was used as template for DNA amplification of clone CM-8.3 by using oligonucleotides K763 (5′-CTTTCTGGTGGCGGTAGCTCACGTCGCTCTGGCG-3′) and K764 (5′-GGTGCTACCGCCACCAGAAAACGATCGTTTCTTTTCGC-3′).

The oligonucleotide K764 contains a sequence encoding for the linker SGGGS, which joins the sequences CM-8.3 and CM-2.10. The PCR protocol was 30″ at 94° C., 30″ at 52° C. and 30″ at 72° C. for 25 cycles.

SEQ ID 2 was used as template for DNA amplification of clone CM-2.10 by using oligonucleotides K765 (5′-TTTTCTGGTGGCGGTAGCACCAAAGACA CGTCGTTAC-3′) and K766 (5′-CCGCGGCCGCTACCGCCACCAGAATGCG-3′). PCR protocol was 30″ at 94° C., 30″ at 52° C. and 30″ at 72° C. for 25 cycles.

The PCR products were purified by means of the “Qiagen Purification Kit” (Qiagen, CA, USA). 50 ng of DNA amplification products of SEQ ID 6 and SEQ ID 8 were mixed together and used as templates in PCR reaction by using oligonucleotides K761 and K764. The PCR protocol was 30″ at 94° C., 30″ at 52° C. and 90″ at 72° C. for 30 cycles. 50 ng of the resulting DNA amplification was purified and then mixed with 50 ng of DNA amplification product of SEQ ID 2. Finally, the DNA mixture was used as template for DNA amplification by using oligonucleotides K761 and K766, following PCR condition of 30″ at 94° C., 30″ at 52° C. and 120″ at 72° C. for 30 cycles.

EC14 protein product is a chimeric molecule containing the DNA sequences of clones CM-2.7, CM-2.10 and CM-1.3.

SEQ ID 12 was used as template for DNA of clone CM-2.7 by using oligonucleotides K825 (5′-GGGGATCCCACTAGTCGTGCTGGCCAGCCG CTG-3′) and K826 (5′-GGTGCTACCGCCACCAGAAAGGTCCCTTAAAGC CCAAAC-3′). The oligonucleotide K826 contains a sequence encoding for the linker SGGGS, which joins the sequences CM-2.7 and CM-2.10.

SEQ ID 2 was used as template for DNA amplification of clone CM-2.10 by using oligonucleotides K827 (5′-CCTTTCTGGTGGCGGTAGCACCAAAG ACACGTCGTTACAG-3′) and K828 (5′-GAGACTACCACCCCCGGAATGCGTG CCAGTCTGTCCG-3′).

SEQ ID 10 was used as template for DNA amplification of clone CM-1.3 by using oligonucleotides K829 (5′-CATTCCGGGGGTGGTAGTCTCACGA GCCAGAAACCGG-3′) and K830 (5′-CCAGACTCGAGTCACCCGCGGCCGC TACCGCCACCAGAGCTGCC-3′).

The PCR products were purified by means of the “Qiagen Purification Kit” (Qiagen, CA, USA). 50 ng of DNA amplification products of SEQ ID 12 and SEQ ID 2 were mixed together and used as templates in PCR reaction by using oligonucleotides K825 and K828. PCR condition was 30″ at 94° C., 30″ at 50° C. and 90″ at 72° C. for 30 cycles. 50 ng of the resulting DNA amplification was purified with “Qiagen Purification Kit” (Qiagen, CA, USA) and then mixed with 50 ng of DNA amplification product of SEQ ID 10. Finally, the DNA mixture was used as template for DNA amplification by using K825 and K830, following PCR condition of 30″ at 94° C., 30″ at 50° C. and 120″ at 72° C. for 30 cycles.

The following Table 2 gives, by way of examples, the DNA sequences of the EC7 and EC8 chimeric antigens:

TABLE 2

| Name | Sequence |
|---|---|
| EC7 | ACTAGTGGCAGTCAGAAACCGACCAGCGGTCCCTTGAACATC |
| (SEQ ID | CCGCAACAACAACAGCGTCACGCGGCTTTCAGTCTCGTCTCC |
| 15) | CCGCAGGTGACCAAGGCCAGCCCGGGAAGGGTCCGTCGGGAC |
| | AGCGCGTGGGACGTGAGGCCGCTCACGGAGACCAGAGGGGAT |
| | CTTTTCTCGGGCGACGAGGATTCCGACAGCTCGGATGGCTAT |
| | CCCCCCAACCGTCAAGATCCGCGTTTCACCGACACGCTGGTG |
| | GACATCACGGATACCGAGATTTCTGGTGGCGGTAGCCTGGTG |
| | GACATCACGGATACCGAGACGAGCGCCAAACCGCCCGTCACC |
| | ACCGCGTACAAGTTCGAGCAACCGACGTTGACGTTCGGCGCC |
| | GGAGTTAACGTTCCTGCTGGCGCCGGCGCTGCCATCCTCACG |
| | CCGACGCCTGTCAATCCTTCCACGGCCCCCGCTCCGGCCCCG |
| | ACACCTACCTTCGCGGGTACCCAAACCCCGGTCAACGGTAAC |
| | TCGCCCTGGGCTCCGACGGCGCCGTTGCCCGGGGATATGAAC |
| | CCCGCCAACTGGCCGCGCGAACGCGCGTGGGCCCTCAAGAAT |
| | CCTCACCTGGCTTACAATCCCTTCAGGATGCCTACGACTTCC |
| | ACGGCTTCTCAAAACACCGTGTCCACCACCCCTCGGAGGCCG |
| | TCGACTCCACGCGCCGCGGTGACACAAACAGCGTCTCGGGAC |
| | GCCGCTGATGAGGTTTGGGCTTTAAGGGACCTTTCTGGTGGC |
| | GGTAGCACGAGCCAGAAACCGGTGCTGGGCAAGCGAGTCGCG |
| | ACGCCGCACGCGTCCGCCCGAGCGCAGACGGTGACGTCGACA |
| | CCGGTTCAGGGAAGGGTAGAGAAACAGGTATCGGGCACGCCG |
| | TCGACGGTACCCGCCACGCTGTTGCAACCTCAACCGGCTTCG |
| | TCTAAAACAACGTCATCAAGGAACGTGACTTCTGGCGCGAGA |
| | ACCTCTTCCGCTTCGGCTCGACAGCCGTCAGCCTCGGCGTCC |
| | GTTTTGTCGCCCACGGAGGATGATGTCGTGTCCCCCTCTGGT |
| | GGCGGTAGCGGCCGC |
| EC8 | ACTAGTGCTCACATTAACACCGTCTCCTGTCCTACCGTTATG |
| (SEQ ID | AGGTTCGACCAGCGGCTGCTGGAAGAGGGCGACGAGGAGGAT |
| 17) | GAAGTGACCGTGATGTCGCCGTCACCCGAGCCCGTGCAACAG |
| | CAGCCGCCGGTCGAGCCCGTGCAGCAGCAGCCCCAGGGACGC |
| | GGGTCTCACCGTCGGCGCTACAAGGAGTCGGCGCCGCAAGAG |
| | ACGCTGCCTACGAATCACGAACGCGAGATTTTGGATCTCATG |
| | CGACACAGCCCCGACGTGCCTCGGGAGGCGGTGATGTCACCG |
| | ACCATGGTCACCATACCTCCTCCCCAGATACCCTTTGTGGGT |
| | TCCGCGCGTGAACTTTCTGGTGGCGGTAGCTCACGTCGCTCT |
| | GGCGAACCCTCGACGGTGATTTATATCCCCTCGAGCAACGAG |
| | GACACGCCGGCGGATGAGGAGGCGGAGGACAGCGTTTTCACG |
| | AGCACGCGGGCGCGCAGCGCCACGGAAGATCTGGATCGCATG |
| | GAGGCCGGTTTGTCGCCCTACAGCGTCTCCTCGGACGCTCCG |
| | TCGTCCTTCGAGCTCGTGCGCGAGACCGGCGGCACCGGCGCC |
| | GCCAAGAAACCGAGCGAAAAGAAACGATCGTTTTCTGGTGGC |
| | GGTAGCACCAAAGACACGTCGTTACAGGCTCCGCCTTCCTAC |
| | GAGGAAAGTGTTTATAATTCTGGTCGCAAAGGACCGGGACCA |
| | CCGTCGTCTGATGCATCCACGGCGGCTCCGCCTTACACCAAC |
| | GAGCAGGCTTACCAGATGCTTCTGGCCCTGGCCCGTCTGGAC |
| | GCAGAGCAGCGAGCGCAGCAAAACGGTACAGATTCTTTGGAC |
| | GGACAGACTGGCACGCATTCTGGTGGCGGTAGCGGCCGC |
| EC14 | ACTAGTCGTGCTGGCCAGCCGCTGGTGGACATCACGGATACC |
| (SEQ ID | GAGACGAGCGCCAAACCGCCCGTCACCACCGCGTACAAGTTC |
| NO: 35) | GAGCAACCGACGTTGACGTTCGGCGCCGGAGTTAACGTTCCT |
| | GCTGGCGCCGGCGCTGCCATCCTCACGCCGACGCCTGTCAAT |
| | CCTTCCACGGCCCCCGCTCCGGCCCCGACACCTACCTTCGCG |
| | GGTACCCAAACCCCGGTCAACGGTAACTCGCCCTGGGCTCCG |
| | ACGGCGCCGTTGCCCGGGGATATGAACCCCGCCAACTGGCCG |
| | CGCGAACGCGCGTGGGCCCTCAAGAATCCTCACCTGGCTTAC |
| | AATCCCTTCAGGATGCCTACGACTCCACGGCTTCTCAAAACA |
| | CCGTGTCCACCACCCCTCGGAGGCCGTCGACTCCACGCGCCG |
| | CGGTGACACAAACAGCGTCTCGGGACGCCGCTGATGAGGTTT |
| | GGGCTTTAAGGGACCTTTCTGGTGGCGGTAGCACCAAAGACA |
| | CGTCGTTACAGGCTCCGCCTTCCTACGAGGAAAGTGTTTATA |
| | ATTCTGGTCGCAAAGGACCGGGACCACCGTCGTCTGATGCAT |
| | CCACGGCGGCTCCGCCTTACACCAACGAGCAGGCTTACCAGA |
| | TGCTTCTGGCCCTGGCCCGTCTGGACGCAGAGCAGCGAGCGC |
| | AGCAAAACGGTACAGATTCTTTGGACGGACAGACTGGCACGC |
| | ATTCCGGGGGTGGTAGTCTCACGAGCCAGAAACCGGTGCTGG |
| | GCAAGCGAGTCGCGACGCCGCACGCGTCCGCCCGAGCGCAGA |
| | CGGTGACGTCGACGCCGGTTCAGGGAAGGCTAGAGAAACAGG |
| | TGTCGGGCACGCCGTCGACGGTACCCGCCACGCTGTTGCAAC |
| | CTCAACCGGCTTCGTCTAAAACGACGTCATCAAGGAACGTGA |
| | CTTCTGGCGCGGGAACCTCTTCCGCTTCTTCGGCTCGACAGC |
| | CGTCAGCCTCGGCGTCCGTTTTGTCGCCCACGGAGGATGATG |
| | TCGTGTCCCCCGCCACATCGCCGCTGTCCATGCTTCGTCAGC |
| | CTCTCCGTCCCCGGCCAAGAGTGCCCCCCCGTCTCCGGTGAA |
| | AGGCCGGGGCAGCCGCGTCGGTGTTCCTTCCTTGAAACCTAC |
| | TTTGGGCGGCAAGGCGGTGGTAGGTCGACCGCCCTCGGTCCC |
| | CGTGAGCGGTAGCGCGCCGGGTCGCCTGTCCGGCAGCTCTGG |
| | TGGCGGTAGCGGCCGC |

The chimeric protein EC7 has the amino acid sequence TSGSQKPTSGPLNIPQQQQRHAAFSLVSPQVTKASPGRVRRDSAWDVRPLTETRG DLFSGDEDSDSSDGYPPNRQDPRFTDTLVDITDTEISGGGSLVDITDTETTAYKFEQ PTLTFGAGVNVPAGAGAAILTPTPVNPSTAPAPAPTPTFAGTQTPVNGNSPWAPTA PLPGDMNPANWPRERAWALKNPHLAYNPFRMPTTSTASQNTVSTTPRRPSTPRAA VTQTASRDAADEVWALRDLSGGGSTSQKPVLGKRVATPHASARAQTVTSTPVQGR VEKQVSGTPSTVPATLLQPQPASSKTTSSRNVTSGARTSSASARQPSASASVLSPT EDDWSPSGGGSGR (SEQ ID 16) and its use as recombinant antigen, containing multiple HCMV protein fragments, is covered by the present invention.

The chimeric protein EC8 has the amino acid sequence TSAHINTVSCPTVMRFDQRLLEEGDEEDEVTVMSPSPEPVQQQPPVEPVQQQPQG RGSHRRRYKESAPQETLPTNHEREILDLMRHSPDVPREAVMSPTMVTIPPPQIPFV GSARELSGGGSSRRSGEPSTVIYIPSSNEDTPADEEAEDSVFTSTRARSATEDLDR MEAGLSPYSVSSDAPSSFELVRETGGTGAAKKPSEKKRSFSGGGSTKDTSLQAPP SYEESVYNSGRKGPGPPSSDASTAAPPYTNEQAYQMLLALARLDAEQRAQQNGTD SLDGQTGTHSGGGSGR (SEQ ID 18) and its use as recombinant antigen, containing multiple HCMV protein fragments, is covered by the present invention.

The chimeric protein EC14 has the amido acid sequence PLVDITDTETSAKPPVTTAYKFEQPTLTFGAGVNVPAGAGAAILTPTPVNPSTAPAPA PTPTFAGTQTPVNGNSPWAPTAPLPGDMNPANWPRERAWALKNPHLAYNPFRMP TTSTASQNTVSTTPRRPSTPRAAVTQTASRDAADEVWALRDLSGGGSTKDTSLQA PPSYEESVYNSGRKGPGPPSSDASTAAPPYTNEQAYQMLLALARLDAEQRAQQNG TDSLDGQTGTHSGGGSLTSQKPVLGKRVATPHASARAQTVTSTPVQGRLEKQVSG TPSTVPATLLQPQPASSKTTSSRNVTSGAGTSSASSARQPSASASVLSPTEDDWS PATSPLSMLSSASPSPAKSAPPSPVKGRGSRVGVPSLKPTLGGKAVVGRPPSVPV SGSAPGRLSGSSGGGSGR (SEQ ID NO: 36) and its use as recombinant antigen, containing multiple HCMV protein fragments, is covered by the present invention.

Construction of DNA Vectors Directing the Expression of Recombinant Antigens as Fusion Products with GST in the Cytoplasm of E. coli Cells

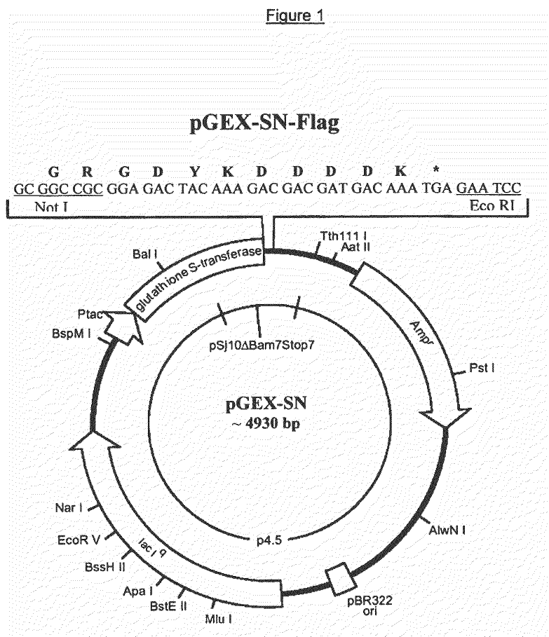

DNA fragments encoding for the selected HCMV phage clones were cloned as fusion products with the protein Glutathione Sulpho Transferase (GST) and expressed as soluble proteins in the cytoplasm of bacterial cells, for the purpose of determining their specificity and selectivity. DNA sequences of clones CM-2.10, CM-3.3, CM-8.3, CM-7.3, CM-1.2, CM-1.3, CM-1.5, CM-2.7, CM-2.11 and CM-4.4 were digested with the restriction enzymes SpeI and NotI. Digested DNA were cloned into vector pGEX-SN (Minenkova et al., International Journal of Cancer, 2003, 106:534-44), which was previously digested with SpeI and NotI endonucleases, to generate fusion products at the carboxy terminus of GST protein. Also, the DNA encoding for the chimeric antigens EC7, EC8 and EC14 were cloned into vectors pGEX-SN and pGEX-SN-Flag to generate GST-fusion products. The plasmid pGEX-SN-Flag was constructed by inserting a short dsDNA sequence obtained by annealing oligonucleotides K718 (5′-GGCCGCGGAGACTACAAAGACGACGATGACAA ATGAG-3′) and K719 (5′-AATTCTCATTTGTCATCGTCGTCTTTGTAGTC TCCGC-3′) into SpeI-NotI digested pGEX-SN vector (see FIG. 1). The resulting plasmids were used to transform competent E. coli cells following standard protocols (Sambrook et al., 1989, Molecular Cloning, Cold Spring Harbor Laboratory Press, Cold Spring Harbor).

### Biochemical Characterisation of Recombinant Antigens

The recombinant GST fusion proteins were expressed in the cytoplasm of transformed E. coli cells and purified by affinity chromatography using Glutathione-Sepharose resin (Amersham Pharmacia Biotech, Sweden), following the manufacturer's instructions. Protein purity and concentration were assessed by SDS-PAGE (Sodium Dodecyl Sulphate-Poly-acrylamide Gel Electrophoresis) analysis and Bradford assay, respectively.

The recombinant chimeric antigens GST-EC7-Flag and GST-EC8-Flag were also subject to Western Blot analysis using an anti-FLAG-M2 monoclonal antibody (1 μg/ml; Sigma-Aldrich, USA) as the primary antibody, an alkaline phosphatase-conjugated goat anti-mouse-IgG antibodies (diluted 1:10000; Sigma-Aldrich, USA) as the secondary antibody, and nitroblue tetrazolium (NBT) plus 5-bromo-4-chloro-3-indosyl phosphate (BCIP) as substrates.

The affinity-purified recombinant products were dialyzed against PBS, diluted at the concentration of 1 mg/ml with PBS and stored at −20° C. until use. The yield of purified products ranged from 4 mg/liter to 15 mg/liter of bacterial culture.

Immunoreactivity of Single Recombinant Antigen Fragments with IgG Antibodies from Sera of HCMV Infected Individuals: IgG Rec-ELISA

The ELISA performance of the GST fusion products was performed by coating Maxisorb-multiwells plates (Nunc) with single antigen fragments at a concentration of 1 μg/ml in coating buffer (50 mM NaHCO₃, pH 9.6). After incubation overnight at 4° C. plates were incubated for 1 h at 37° C. with blocking buffer (5% non-fat dry milk, 0.05% Tween-20 in PBS) and subsequently incubated for 1 h at 37° C. with sera from HCMV-seropositive and seronegative individuals, diluted 1:100 in blocking solution. The plates were extensively washed with 0.05% Tween-20 in PBS and anti-human-IgG alkaline phosphatase-conjugated antibodies (Sigma-Aldrich, USA) diluted 1:7500 in blocking solution were then added to each well. After 30 min at 37° C. the plates were washed and incubated with the chromogenic substrate p-nitrophenyl phosphate (pNPP; Sigma-Aldrich, USA) in developing solution (10% diethanolamine pH 9.8, 0.5 mM MgCl₂, 0.05% NaN₃). Results were recorded as the difference between the optical density (OD) at 405 nm and 620 nm using an automated ELISA reader (Multiskan Labsystems, Finland). For each serum sample the assay was done in duplicate and average values were calculated.

The following Table 3 summarizes the results of the ELISA assays based on single antigen fragments, expressed as GST fusion proteins, employing serum samples from 36 HCMV-seropositive and 33 HCMV-seronegative individuals.

Determination of HCMV-specific IgG in serum samples were done by the whole-cell, HCMV antigen assay ETI-CYTOK-G PLUS (Diasorin, Saluggia, Italy) in accordance to the manufacturer's instructions. For every recombinant antigen the cutoff value was determined as the mean plus three times the standard deviation of the absorbency readings obtained from the HCMV IgG negative sera. As a control, the IgG reactivity against wild-type GST protein was assessed for each serum. The diagnostic criterion used to assign a positive IgG reactivity against single recombinant antigens was an OD_{GST-antigen} greater than the cutoff and an OD_{GST-antigen} greater than the OD_{GST}. In each column of Table 3 are reported the number and the corresponding percentages of reactive sera.

TABLE 3

| | Sera from HCMV | Sera from HCMV |
|---|---|---|
| Recombinant antigen | infected subjects | uninfected subjects |
| GST-CM1.4 | 12/15 (80%) | 2/15 |
| GST-CM4.4 | 30/36 (83%) | 0/33 |
| GST-CM1.2 | 11/15 (73%) | 0/15 |
| GST-CM2.7 | 35/36 (97%) | 0/33 |
| GST-CM1.5 | 7/15 (47%) | 0/15 |
| GST-CM2.11 | 10/15 (67%) | 2/15 |
| GST-CM1.3 | 35/36 (97%) | 0/33 |
| GST-CM2.10 | 30/36 (83%) | 0/33 |
| GST-CM3.3 | 23/36 (64%) | 1/33 |
| GST-CM8.3 | 15/36 (42%) | 0/33 |
| GST-CM7.3 | 19/36 (53%) | 0/33 |
| GST (wild type) | 0/36 | 0/33 |

The following Table 4 shows the results of the IgG Rec-ELISA assays employing serum samples (CA1-CA25) from 25 women who have had a recent HCMV reactivation or a secondary HCMV infection during pregnancy. Determination of HCMV-specific IgG in serum samples were done by the whole-cell, HCMV antigen assay ETI-CYTOK-G PLUS (Diasorin, Saluggia, Italy) or by using single recombinant antigen immunoassays. For each GST-fusion product the cut-off was determined as the mean plus 3SD of the absorbency readings obtained with sera from HCMV seronegative subjects (n=20). Cut-off values for ETI-CYTO-K PLUS, GST-CM2.10, GST-CM3.3, GST-CM8.3, GST-CM7.3, GST-CM1.3, GST-CM2.7 and GST-CM4.4 were 0.2, 0.073, 0.078, 0.101, 0.089, 0.145, 0.147 and 0.138, respectively. Values typed in bold indicate a positive response. Please note that the numerical values of Optical Density obtained with the standard assay cannot be compared with the others, because all of the assays have been performed without a reference to an International Standard.

TABLE 4

| Serum | CYTOK | IgG Rec-ELISA (Optical Density) | | | | | | |
|---|---|---|---|---|---|---|---|---|
| samples | G PLUS | CM1.3 | CM2.7 | CM4.4 | CM2.10 | CM3.3 | CM8.3 | CM7.3 |
| CA1 | 1.210 | 1.908 | 3.165 | 1.602 | 0.592 | 2.103 | 1.727 | 0.846 |
| CA2 | 0.785 | 0.727 | 3.298 | 0.128 | 0.218 | 0.095 | 0.049 | 0.066 |
| CA3 | 0.566 | 0.524 | 0.389 | 0.157 | 0.280 | 0.299 | 0.164 | 0.313 |
| CA4 | 0.665 | 1.008 | 0.861 | 0.142 | 0.515 | 0.216 | 0.061 | 0.118 |
| CA5 | 0.420 | 0.245 | 0.125 | 0.089 | 0.177 | 0.123 | 0.082 | 0.119 |
| CA6 | 0.798 | 0.697 | 1.197 | 0.340 | 2.046 | 0.642 | 0.060 | 0.081 |
| CA7 | 0.787 | 2.300 | 0.711 | 0.213 | 2.418 | 0.145 | 0.066 | 0.386 |
| CA8 | 1.253 | 1.445 | 1.173 | 0.317 | 2.961 | 0.508 | 0.074 | 0.120 |
| CA9 | 1.099 | 2.231 | 3.031 | 0.689 | 1.942 | 0.238 | 0.131 | 0.303 |
| CA10 | 0.644 | 0.142 | 0.726 | 0.114 | 1.357 | 0.275 | 0.054 | 0.062 |
| CA11 | 1.601 | 2.896 | 3.177 | 0.371 | 3.086 | 1.947 | 1.268 | 1.711 |
| CA12 | 0.937 | 3.196 | 0.438 | 0.340 | 2.592 | 0.150 | 0.078 | 0.071 |
| CA13 | 1.148 | 2.467 | 2.720 | 0.197 | 3.021 | 0.203 | 0.055 | 0.060 |
| CA14 | 1.270 | 0.382 | 0.157 | 0.152 | 2.379 | 0.394 | 0.064 | 0.110 |
| CA15 | 0.585 | 0.257 | 0.426 | 0.059 | 0.846 | 0.071 | 0.064 | 0.085 |
| CA16 | 0.758 | 0.119 | 1.057 | 0.075 | 2.867 | 0.075 | 0.060 | 0.061 |
| CA17 | 0.768 | 2.832 | 0.359 | 0.155 | 2.947 | 0.357 | 0.080 | 0.076 |
| CA18 | 0.816 | 2.393 | 1.641 | 0.395 | 2.633 | 0.675 | 0.069 | 0.138 |
| CA19 | 0.729 | 2.637 | 2.070 | 0.466 | 0.962 | 1.192 | 0.123 | 0.204 |
| CA20 | 0.915 | 2.030 | 1.384 | 0.263 | 2.063 | 0.828 | 0.125 | 0.286 |
| CA21 | 1.076 | 1.246 | 1.496 | 0.141 | 2.794 | 0.390 | 0.077 | 0.081 |
| CA22 | 1.205 | 3.117 | 2.748 | 0.395 | 3.050 | 2.382 | 0.103 | 0.058 |
| CA23 | 0.930 | 3.191 | 2.310 | 1.154 | 1.962 | 0.631 | 0.110 | 0.077 |
| CA24 | 0.856 | 1.722 | 3.046 | 0.355 | 2.815 | 0.445 | 0.065 | 0.152 |
| CA25 | 1.359 | 2.904 | 2.887 | 2.313 | 2.692 | 1.815 | 1.267 | 0.356 |

The following Table 5 shows the performance characteristics of the commercial assay (ETI-CYTOK-G PLUS), compared to the results obtained with single recombinant antigens (IgG Rec-ELISA). From Table 5 it clearly results that, with the exception of the Rec-ELISA based on the CMV-3.3 antigen fragment, both specificity and positive predictive values of the assays (see the 3^{rd} and the 5^{th} column reporting the occurrence of false positives) reached the maximum (100%) when using the recombinant antigen fragments of the invention.

TABLE 5

| | | | Agree- | | |
|---|---|---|---|---|---|
| | Sensitivity | Specificity | ment | PPV* | NPV* |
| Diagnostic test | (%) | (%) | (%) | (%) | (%) |
| ETI-CYTOK-G PLUS | 100 | 100 | 100 | 100 | 100 |
| CM1.3 Rec-ELISA | 92.0 | 100 | 96.6 | 100 | 94.3 |
| CM2.7 Rec-ELISA | 96.0 | 100 | 98.3 | 100 | 97.1 |
| CM4.4 Rec-ELISA | 88.0 | 100 | 94.8 | 100 | 91.7 |
| CM2.10 Rec-ELISA | 100 | 100 | 100 | 100 | 100 |
| CM3.3 Rec-ELISA | 92.0 | 97.1 | 94.8 | 95.8 | 94.1 |
| CM8.3 Rec-ELISA | 36.0 | 100 | 72.4 | 100 | 67.3 |
| CM7.3 Rec-ELISA | 56.0 | 100 | 81.0 | 100 | 75.0 |

*PPV, positive predictive value; NPV, negative predictive value.

Immunoreactivity of the Recombinant Chimeric Antigens with IgG Antibodies from Sera of HCMV Infected Individuals

The ELISA performance of the recombinant chimeric antigens was performed by coating Maxisorb plates (Nunc) with GST-EC7-Flag, GST-EC8-Flag and GST-EC14 at a concentration of 0.5 μg/ml, 2 μg/ml and 3 μg/ml in coating buffer, respectively. After incubation overnight at 4° C. plates were incubated for 1 h at 37° C. with blocking buffer (5% non-fat dry milk, 0.05% Tween-20 in PBS) and then incubated for 1 h at 37° C. with serum samples diluted 1:100 in blocking solution. The plates were washed with 0.05% Tween-20 in PBS and anti-human-IgG horse-radish peroxidase-conjugated antibodies (1 mg/ml; Sigma-Aldrich, USA) diluted 1:20000 in blocking solution were added to each well. Finally, incubating plates with the chromogenic substrate tetramethylbenzidine (TMB; Sigma-Aldrich, USA) revealed the enzymatic activity. Results were recorded as the difference between the absorbance (Optical Density, OD) at 450 and 620 nm, detected by an automated ELISA reader (Labsystem Multiskan, Finland). For each serum sample the assay was done in duplicate and average values were calculated.

The following Table 6 shows the results of the ELISA assays using either the chimeric antigens EC7-Flag, EC8-Flag and GST-EC14 or the whole-cell, HCMV antigen assay ETI-CYTOK-G PLUS (Diasorin, Saluggia, Italy) with serum samples from 36 HCMV-seropositive (C1-C36) and 33 HCMV-seronegative (N1-N33) individuals. For each chimeric antigen the cut-off was determined as the mean plus 3SD of the absorbency readings obtained with sera from HCMV seronegative subjects. Cut-off values for ETI-CYTO-K PLUS, GST-EC7-Flag, GST-EC8-Flag and GST-EC14 were 0.296, 0.296, 0.273 and 0.203, respectively. Values typed in bold indicate a positive response. Please note that the numerical values of Optical Density obtained with the standard assay cannot be compared with the others, because all of the assays have been performed without a reference to an International Standard.

TABLE 6

ELISA ASSAYS (Optical Density)

| Serum | ETI-CYTOK | GST-EC7- | GST-EC8- | GST- |
|---|---|---|---|---|
| samples | G PLUS | Flag | Flag | EC14 |
| C1 | 0.805 | 1.245 | 0.626 | 0.746 |
| C2 | 1.679 | 2.867 | 2.006 | 2.132 |
| C3 | 1.461 | 2.920 | 1.687 | 2.455 |
| C4 | 2.896 | 2.813 | 2.185 | 2.988 |
| C5 | 1.371 | 2.152 | 1.859 | 1.226 |
| C6 | 2.360 | 2.987 | 2.337 | 3.018 |
| C7 | 2.260 | 2.206 | 1.514 | 2.153 |
| C8 | 2.585 | 1.579 | 2.771 | 1.405 |
| C9 | 1.975 | 2.622 | 2.421 | 2.340 |
| C10 | 0.961 | 2.500 | 2.499 | 2.741 |
| C11 | 1.385 | 1.455 | 1.480 | 1.411 |
| C12 | 0.657 | 0.595 | 1.825 | 1.206 |
| C13 | 0.624 | 1.049 | 1.161 | 1.671 |
| C14 | 1.171 | 1.543 | 1.044 | 0.899 |
| C15 | 0.576 | 1.319 | 0.386 | 1.092 |
| C16 | 2.772 | 2.927 | 2.225 | 3.104 |
| C17 | 2.115 | 3.002 | 2.127 | 2.831 |
| C18 | 2.019 | 2.541 | 2.313 | 1.468 |
| C19 | 2.137 | 1.514 | 2.421 | 2.045 |
| C20 | 0.637 | 0.814 | 1.475 | 1.097 |
| C21 | 2.638 | 3.081 | 1.706 | 2.996 |
| C22 | 0.622 | 1.222 | 1.157 | 1.011 |
| C23 | 3.028 | 3.054 | 1.717 | 3.201 |
| C24 | 1.027 | 1.831 | 1.863 | 0.935 |
| C25 | 1.333 | 2.001 | 1.738 | 1.146 |
| C26 | 0.668 | 1.101 | 1.162 | 1.327 |
| C27 | 1.756 | 2.793 | 1.005 | 1.526 |
| C28 | 0.759 | 1.323 | 0.822 | 0.828 |
| C29 | 1.502 | 2.815 | 2.176 | 2.613 |
| C30 | 1.869 | 0.340 | 1.011 | 2.241 |
| C31 | 1.208 | 1.245 | 0.653 | 0.917 |
| C32 | 0.523 | 1.732 | 0.492 | 1.523 |
| C33 | 0.907 | 0.515 | 0.322 | 0.424 |
| C34 | 0.737 | 2.603 | 1.859 | 2.242 |
| C35 | 0.958 | 2.761 | 1.606 | 2.302 |
| C36 | 0.281 | 1.270 | 0.486 | 0.254 |
| N1 | 0.087 | 0.188 | 0.141 | 0.073 |
| N2 | 0.174 | 0.164 | 0.084 | 0.076 |
| N3 | 0.129 | 0.185 | 0.149 | 0.075 |
| N4 | 0.121 | 0.081 | 0.064 | 0.069 |
| N5 | 0.057 | 0.214 | 0.145 | 0.040 |
| N6 | 0.273 | 0.118 | 0.049 | 0.186 |
| N7 | 0.095 | 0.143 | 0.107 | 0.051 |
| N8 | 0.092 | 0.182 | 0.119 | 0.054 |
| N9 | 0.090 | 0.097 | 0.176 | 0.132 |
| N10 | 0.119 | 0.103 | 0.062 | 0.085 |
| N11 | 0.089 | 0.144 | 0.031 | 0.055 |
| N12 | 0.093 | 0.208 | 0.047 | 0.040 |
| N13 | 0.080 | 0.077 | 0.058 | 0.169 |
| N14 | 0.084 | 0.154 | 0.112 | 0.038 |
| N15 | 0.105 | 0.130 | 0.086 | 0.051 |
| N16 | 0.114 | 0.086 | 0.139 | 0.146 |
| N17 | 0.178 | 0.058 | 0.015 | 0.049 |
| N18 | 0.276 | 0.212 | 0.302 | 0.037 |
| N19 | 0.118 | 0.075 | 0.067 | 0.043 |
| N20 | 0.118 | 0.110 | 0.113 | 0.049 |
| N21 | 0.172 | 0.191 | 0.132 | 0.052 |
| N22 | 0.088 | 0.066 | 0.055 | 0.041 |
| N23 | 0.103 | 0.244 | 0.328 | 0.112 |
| N24 | 0.136 | 0.160 | 0.127 | 0.101 |
| N25 | 0.085 | 0.091 | 0.088 | 0.095 |
| N26 | 0.096 | 0.186 | 0.073 | 0.034 |
| N27 | 0.096 | 0.116 | 0.099 | 0.045 |
| N28 | 0.071 | 0.107 | 0.102 | 0.162 |
| N29 | 0.100 | 0.108 | 0.052 | 0.056 |
| N30 | 0.047 | 0.065 | 0.039 | 0.072 |
| N31 | 0.167 | 0.212 | 0.139 | 0.123 |
| N32 | 0.069 | 0.072 | 0.077 | 0.064 |
| N33 | 0.074 | 0.096 | 0.129 | 0.077 |

The following Table 7 shows the performance characteristics of the commercial assay (ETI-CYTOK-G PLUS), compared to the results obtained with the EC7 and EC8 chimeric antigens (IgG Rec-ELISA). From Table 7 it clearly results that the sensitivity of the assay (see the 2^{nd} column reporting the occurrence of false negatives) reaches the maximal value when using the chimeric antigens of the invention. Also, it should be noted that both the commercial test ETI-CYTOK-G employing the lysed, whole-cell CMV antigen and the IgG rec-ELISA with the chimeric antigens EC7 or EC14 display identical performance characteristics, while keeping the reproducibility levels typically associated with assays carried out with recombinant antigens.

TABLE 7

| | Sensitivity | Specificity | Agreement | PPV* | NPV* |
|---|---|---|---|---|---|
| Diagnostic test | (%) | (%) | (%) | (%) | (%) |
| ETI-CYTOK-G | 100 | 100 | 100 | 100 | 100 |
| PLUS | | | | | |
| EC7-Flag IgG | 100 | 100 | 100 | 100 | 100 |
| Rec-ELISA | | | | | |
| EC8-Flag IgG | 100 | 97.1 | 98.6 | 97.3 | 100 |
| Rec-ELISA | | | | | |
| EC14 IgG Rec- | 100 | 100 | 100 | 100 | 100 |
| ELISA | | | | | |

*PPV, positive predictive value; NPV, negative predictive value.

1. A chimeric recombinant antigen containing the fusion of at least three different antigenic regions of HCMV proteins, wherein said antigenic regions are B-cell epitopes, which bind to HCMV-specific antibodies, and wherein one of the three different antigenic regions consists of the amino acid sequence of: SEQ ID NO: 2 or SEQ ID NO: 12.

2. The chimeric antigen of claim 1, wherein the HCMV-specific antibodies are extracted from sera of subjects who have been infected by HCMV.

3. The chimeric antigen of claim 1, wherein the three different antigenic regions are linked by a covalent bond or by a peptide linker.

4. The chimeric antigen of claim 1, wherein said chimeric antigen contains both amino acid sequences of SEQ ID NO: 2 and SEQ ID NO: 12.

5. The chimeric antigen of claim 1, wherein said chimeric antigen further contains the amino acid sequence of SEQ ID NO: 10.

6. The chimeric antigen of claim 1, wherein said chimeric antigen further contains the amino acid sequence of SEQ ID NO: 14.

7. The chimeric antigen of claim 1, wherein said chimeric antigen further contains an amino acid sequence selected from the group consisting of: SEQ ID NO: 4, SEQ ID NO: 6 and SEQ ID NO: 8.

8. The chimeric antigen of claim 1, comprising the amino acid sequence of SEQ ID NO: 16.

9. The chimeric antigen of claim 1, comprising the amino acid sequence of SEQ ID NO: 18.

10. The chimeric antigen of claim 1, the chimeric antigen comprising the amino acid sequence of SEQ ID NO: 36.

11. A nucleotide sequence coding for the antigen according to claim 1.

12. The nucleotide sequence according to claim 11, the nucleotide sequence comprising the nucleotide sequence of SEQ ID NO: 15.

13. The nucleotide sequence according to claim 11, the nucleotide sequence comprising the nucleotide sequence of SEQ ID NO: 17.

14. The nucleotide sequence according to claim 11, the nucleotide sequence comprising the nucleotide sequence of SEQ ID NO: 35.

15. A nucleotide sequence that hybridizes with any sequence according to claim 11 under stringent hybridization conditions.

16. The chimeric recombinant antigen encoded by the nucleotide sequence of claim 15.

17. The nucleotide sequence of claim 11, which is a DNA sequence.

18. A vector comprising the DNA sequence of claim 17.

19. A host cell transformed with the vector of claim 18.

20. A process for the production of the antigen according to claim 1, comprising
culturing the host cell transformed with a vector comprising a DNA nucleotide sequence coding for the antigen of claim 1 to provide a desired product comprising the antigen of claim 1; and
isolating the desired product.

21. An active agent for the diagnosis of HCMV infections, the active agent comprising the antigen of claim 1.

22. A diagnostic agent for detecting HCMV infections, the diagnostic agent comprising at least one antigen according to claim 1.

23. An assay kit for diagnosis of a HCMV infection, the assay kit containing at least one diagnostic agent according to claim 22.

24. A method for diagnosis of a HCMV infection, the method comprising
contacting a test sample with the diagnostic agent according to claim 22.

25. The method according to claim 24, wherein the test sample is examined for presence of HCMV antibodies and wherein the method comprises the steps of:
(i) incubating the test sample with a diagnostic agent comprising at least one chimeric recombinant antigen containing the fusion of at least three different antigenic regions of HCMV proteins, wherein said antigenic regions are B-cell epitopes, which bind to HCMV-specific antibodies, and wherein one of the three different antigenic regions consists of the amino acid sequence of: SEQ ID NO: 2 or SEQ ID NO: 12,
(ii) allowing formation of an antibody-diagnostic agent complex, and
(iii) detecting the presence of the antibody-diagnostic agent complex.

26. The method according to claim 25, wherein the test sample is serum or plasma of a subject suspected of being infected with HCMV.

27. A medicament comprising the antigen according to claim 1.

28. A process for preparation of a medicament for prevention or treatment of HCMV infections, the process comprising
mixing the antigen of claim 1 with a pharmaceutically acceptable carrier.

29. A medicament comprising the nucleotide sequence of claim 11.

30. A process for the preparation of a medicament useful for the treatment or prevention of HCMV infections the process comprising
mixing the nucleotide sequence of claim 11 with a pharmaceutically acceptable carrier.

31. An immunomodulatory vaccine comprising at least one antigen according to claim 1 together with an adjuvant.

32. The vaccine according to claim 31, wherein the adjuvant is selected from the group consisting of an aluminium salt and an oil in water emulsion.

33. A pharmaceutical composition, containing at least one antigen according to claim 1.

34. A pharmaceutical composition, containing at least one nucleotide sequence according to claim 11.

35. The composition according to claim 33, wherein the composition is suitable for human and/or veterinary use.

36. A method of treating a mammal for a HCMV infection, the method comprising
administering to the mammal a therapeutically effective amount of the vaccine of claim 31.

37. A method for treating or preventing a HCMV infection in a mammal, the method comprising administering to the mammal the medicament according to claim 27.

38. A method for treating or preventing a HCMV infection in a mammal, the method comprising administering to the mammal the medicament according to claim 29.

39. The pharmaceutical composition according to claim 33, wherein the composition is in the form of a vaccine.

40. The pharmaceutical composition according to claim 34, wherein the composition is in the form of a vaccine.


SEQUENCE LISTING (42 sequences)

SEQ ID NO: 1 - DNA, 222 bp, Human cytomegalovirus
  CDS (1)..(222)
accaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcaaaacggtacagattctttggacggacagactggcacgcat

SEQ ID NO: 2 - protein, 74 aa, Human cytomegalovirus
TKDTSLQAPPSYEESVYNSGRKGPGPPSSDASTAAPPYTNEQAYQMLLALARLDAEQRAQQNGTDSLDGQTGTH

SEQ ID NO: 3 - DNA, 345 bp, Human cytomegalovirus
  CDS (1)..(345)
gctcacattaacaccgtctcctgtcctaccgttatgaggttcgaccagcggctgctggaagagggcgacgaggaggatgaagtgaccgtgatgtcgccgtcacccgagcccgtgcaacagcagccgccggtcgagcccgtgcagcagcagccccagggacgcgggtctcaccgtcggcgctacaaggagtcggcgccgcaagagacgctgcctacgaatcacgaacgcgagattttggatctcatgcgacacagccccgacgtgcctcgggaggcggtgatgtcaccgaccatggtcaccatacctcctccccagataccctttgtgggttccgcgcgtgaactt

SEQ ID NO: 4 - protein, 115 aa, Human cytomegalovirus
AHINTVSCPTVMRFDQRLLEEGDEEDEVTVMSPSPEPVQQQPPVEPVQQQPQGRGSHRRRYKESAPQETLPTNHEREILDLMRHSPDVPREAVMSPTMVTIPPPQIPFVGSAREL

SEQ ID NO: 5 - DNA, 255 bp, Human cytomegalovirus
  CDS (1)..(255)
tcacgtcgctctggcgaaccctcgacggtgatttatatcccctcgagcaacgaggacacgccggcggatgaggaggcggaggacagcgttttcacgagcacgcgggcgcgcagcgccacggaagatctggatcgcatggaggccggtttgtcgccctacagcgtctcctcggacgctccgtcgtccttcgagctcgtgcgcgagaccggcggcaccggcgccgccaagaaaccgagcgaaaagaaacgatcgttt

SEQ ID NO: 6 - protein, 85 aa, Human cytomegalovirus
SRRSGEPSTVIYIPSSNEDTPADEEAEDSVFTSTRARSATEDLDRMEAGLSPYSVSSDAPSSFELVRETGGTGAAKKPSEKKRSF

SEQ ID NO: 7 - DNA, 96 bp, Human cytomegalovirus
  CDS (1)..(96)
cttattctcgaggagattcgacgtccgctgccagatggcacggggggcgacggccccgagggcgaggctattcacctgcgtggacgggaggcgcat

SEQ ID NO: 8 - protein, 32 aa, Human cytomegalovirus
LILEEIRRPLPDGTGGDGPEGEAIHLRGREAH

SEQ ID NO: 9 - DNA, 480 bp, Human cytomegalovirus
  CDS (1)..(480)
acgagccagaaaccggtgctgggcaagcgagtcgcgacgccgcacgcgtccgcccgagcgcagacggtgacgtcgacgccggttcagggaaggctagagaaacaggtgtcgggcacgccgtcgacggtacccgccacgctgttgcaacctcaaccggcttcgtctaaaacgacgtcatcaaggaacgtgacttctggcgcgggaacctcttccgcttcttcggctcgacagccgtcagcctcggcgtccgttttgtcgcccacggaggatgatgtcgtgtcccccgccacatcgccgctgtccatgctttcgtcagcctctccgtccccggccaagagtgcccccccgtctccggtgaaaggccggggcagccgcgtcggtgttccttccttgaaacctactttgggcggcaaggcggtggtaggtcgaccgccctcggtccccgtgagcggtagcgcgccgggtcgcctgtccggcagc

SEQ ID NO: 10 - protein, 160 aa, Human cytomegalovirus
TSQKPVLGKRVATPHASARAQTVTSTPVQGRLEKQVSGTPSTVPATLLQPQPASSKTTSSRNVTSGAGTSSASSARQPSASASVLSPTEDDVVSPATSPLSMLSSASPSPAKSAPPSPVKGRGSRVGVPSLKPTLGGKAVVGRPPSVPVSGSAPGRLSGS

SEQ ID NO: 11 - DNA, 462 bp, Human cytomegalovirus
  CDS (1)..(462)
ccgctggtggacatcacggataccgagacgagcgccaaaccgcccgtcaccaccgcgtacaagttcgagcaaccgacgttgacgttcggcgccggagttaacgttcctgctggcgccggcgctgccatcctcacgccgacgcctgtcaatccttccacggcccccgctccggccccgacacctaccttcgcgggtacccaaaccccggtcaacggtaactcgccctgggctccgacggcgccgttgcccggggatatgaaccccgccaactggccgcgcgaacgcgcgtgggccctcaagaatcctcacctggcttacaatcccttcaggatgcctacgacttccacggcttctcaaaacaccgtgtccaccacccctcggaggccgtcgactccacgcgccgcggtgacacaaacagcgtctcgggacgccgctgatgaggtttgggctttaagggacctt

SEQ ID NO: 12 - protein, 154 aa, Human cytomegalovirus
PLVDITDTETSAKPPVTTAYKFEQPTLTFGAGVNVPAGAGAAILTPTPVNPSTAPAPAPTPTFAGTQTPVNGNSPWAPTAPLPGDMNPANWPRERAWALKNPHLAYNPFRMPTTSTASQNTVSTTPRRPSTPRAAVTQTASRDAADEVWALRDL

SEQ ID NO: 13 - DNA, 270 bp, Human cytomegalovirus
  CDS (1)..(270)
ccgggcagtcagaaaccgaccagcggtcccttgaacatcccgcaacaacaacagcgtcacgcggctttcagtctcgtctccccgcaggtgaccaaggccagcccgggaagggtccgtcgggacagcgcgtgggacgtgaggccgctcacggagaccagaggggatcttttctcgggcgacgaggattccgacagctcggatggctatccccccaaccgtcaagatccgcgtttcaccgacacgctggtggacatcacggataccgagatt

SEQ ID NO: 14 - protein, 90 aa, Human cytomegalovirus
PGSQKPTSGPLNIPQQQQRHAAFSLVSPQVTKASPGRVRRDSAWDVRPLTETRGDLFSGDEDSDSSDGYPPNRQDPRFTDTLVDITDTEI

SEQ ID NO: 15 - DNA, 1065 bp, Artificial
  Synthetic
actagtggcagtcagaaaccgaccagcggtcccttgaacatcccgcaacaacaacagcgtcacgcggctttcagtctcgtctccccgcaggtgaccaaggccagcccgggaagggtccgtcgggacagcgcgtgggacgtgaggccgctcacggagaccagaggggatcttttctcgggcgacgaggattccgacagctcggatggctatccccccaaccgtcaagatccgcgtttcaccgacacgctggtggacatcacggataccgagatttctggtggcggtagcctggtggacatcacggataccgagacgagcgccaaaccgcccgtcaccaccgcgtacaagttcgagcaaccgacgttgacgttcggcgccggagttaacgttcctgctggcgccggcgctgccatcctcacgccgacgcctgtcaatccttccacggcccccgctccggccccgacacctaccttcgcgggtacccaaaccccggtcaacggtaactcgccctgggctccgacggcgccgttgcccggggatatgaaccccgccaactggccgcgcgaacgcgcgtgggccctcaagaatcctcacctggcttacaatcccttcaggatgcctacgacttccacggcttctcaaaacaccgtgtccaccacccctcggaggccgtcgactccacgcgccgcggtgacacaaacagcgtctcgggacgccgctgatgaggtttgggctttaagggacctttctggtggcggtagcacgagccagaaaccggtgctgggcaagcgagtcgcgacgccgcacgcgtccgcccgagcgcagacggtgacgtcgacaccggttcagggaagggtagagaaacaggtatcgggcacgccgtcgacggtacccgccacgctgttgcaacctcaaccggcttcgtctaaaacaacgtcatcaaggaacgtgacttctggcgcgagaacctcttccgcttcggctcgacagccgtcagcctcggcgtccgttttgtcgcccacggaggatgatgtcgtgtccccctctggtggcggtagcggccgc

SEQ ID NO: 16 - protein, 355 aa, Artificial
  Synthetic Construct
TSGSQKPTSGPLNIPQQQQRHAAFSLVSPQVTKASPGRVRRDSAWDVRPLTETRGDLFSGDEDSDSSDGYPPNRQDPRFTDTLVDITDTEISGGGSLVDITDTETSAKPPVTTAYKFEQPTLTFGAGVNVPAGAGAAILTPTPVNPSTAPAPAPTPTFAGTQTPVNGNSPWAPTAPLPGDMNPANWPRERAWALKNPHLAYNPFRMPTTSTASQNTVSTTPRRPSTPRAAVTQTASRDAADEVWALRDLSGGGSTSQKPVLGKRVATPHASARAQTVTSTPVQGRVEKQVSGTPSTVPATLLQPQPASSKTTSSRNVTSGARTSSASARQPSASASVLSPTEDDVVSPSGGGSGR

SEQ ID NO: 17 - DNA, 879 bp, Artificial
  Synthetic
actagtgctcacattaacaccgtctcctgtcctaccgttatgaggttcgaccagcggctgctggaagagggcgacgaggaggatgaagtgaccgtgatgtcgccgtcacccgagcccgtgcaacagcagccgccggtcgagcccgtgcagcagcagccccagggacgcgggtctcaccgtcggcgctacaaggagtcggcgccgcaagagacgctgcctacgaatcacgaacgcgagattttggatctcatgcgacacagccccgacgtgcctcgggaggcggtgatgtcaccgaccatggtcaccatacctcctccccagataccctttgtgggttccgcgcgtgaactttctggtggcggtagctcacgtcgctctggcgaaccctcgacggtgatttatatcccctcgagcaacgaggacacgccggcggatgaggaggcggaggacagcgttttcacgagcacgcgggcgcgcagcgccacggaagatctggatcgcatggaggccggtttgtcgccctacagcgtctcctcggacgctccgtcgtccttcgagctcgtgcgcgagaccggcggcaccggcgccgccaagaaaccgagcgaaaagaaacgatcgttttctggtggcggtagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcaaaacggtacagattctttggacggacagactggcacgcattctggtggcggtagcggccgc

SEQ ID NO: 18 - protein, 293 aa, Artificial
  Synthetic Construct
TSAHINTVSCPTVMRFDQRLLEEGDEEDEVTVMSPSPEPVQQQPPVEPVQQQPQGRGSHRRRYKESAPQETLPTNHEREILDLMRHSPDVPREAVMSPTMVTIPPPQIPFVGSARELSGGGSSRRSGEPSTVIYIPSSNEDTPADEEAEDSVFTSTRARSATEDLDRMEAGLSPYSVSSDAPSSFELVRETGGTGAAKKPSEKKRSFSGGGSTKDTSLQAPPSYEESVYNSGRKGPGPPSSDASTAAPPYTNEQAYQMLLALARLDAEQRAQQNGTDSLDGQTGTHSGGGSGR

SEQ ID NO: 19 - DNA, 27 bp, Artificial
  Synthetic
ggactagtggcagtcagaaaccgacca

SEQ ID NO: 20 - DNA, 28 bp, Artificial
  Synthetic
ggactagtggcagtcagaaaccgaccag

SEQ ID NO: 21 - DNA, 35 bp, Artificial
  Synthetic
tctggtggcggtagcctggtggacatcacggatac

SEQ ID NO: 22 - DNA, 39 bp, Artificial
  Synthetic
cgtgctaccgccaccagaaaggtcccttaaagcccaaac

SEQ ID NO: 23 - DNA, 36 bp, Artificial
  Synthetic
tctggtggcggtagcacgagccagaaaccggtgctg

SEQ ID NO: 24 - DNA, 30 bp, Artificial
  Synthetic
ccgcggccgctggacacgacatcatcctcc

SEQ ID NO: 25 - DNA, 28 bp, Artificial
  Synthetic
ggactagtgctcacattaacaccgtctc

SEQ ID NO: 26 - DNA, 36 bp, Artificial
  Synthetic
gtgagctaccgccaccagaaagttcacgcgcggaac

SEQ ID NO: 27 - DNA, 34 bp, Artificial
  Synthetic
ctttctggtggcggtagctcacgtcgctctggcg

SEQ ID NO: 28 - DNA, 38 bp, Artificial
  Synthetic
ggtgctaccgccaccagaaaacgatcgtttcttttcgc

SEQ ID NO: 29 - DNA, 37 bp, Artificial
  Synthetic
ttttctggtggcggtagcaccaaagacacgtcgttac

SEQ ID NO: 30 - DNA, 28 bp, Artificial
  Synthetic
ccgcggccgctaccgccaccagaatgcg

SEQ ID NO: 31 - DNA, 37 bp, Artificial
  Synthetic
ggccgcggagactacaaagacgacgatgacaaatgag

SEQ ID NO: 32 - DNA, 37 bp, Artificial
  Synthetic
aattctcatttgtcatcgtcgtctttgtagtctccgc

SEQ ID NO: 33 - DNA, 44 bp, Artificial
  Synthetic
gcggccgcggagactacaaagacgacgatgacaaatgagaatcc

SEQ ID NO: 34 - protein, 11 aa, Artificial
  Synthetic Construct
GRGDYKDDDDK

SEQ ID NO: 35 - DNA, 1236 bp, Artificial
  Synthetic
actagtcgtgctggccagccgctggtggacatcacggataccgagacgagcgccaaaccgcccgtcaccaccgcgtacaagttcgagcaaccgacgttgacgttcggcgccggagttaacgttcctgctggcgccggcgctgccatcctcacgccgacgcctgtcaatccttccacggcccccgctccggccccgacacctaccttcgcgggtacccaaaccccggtcaacggtaactcgccctgggctccgacggcgccgttgcccggggatatgaaccccgccaactggccgcgcgaacgcgcgtgggccctcaagaatcctcacctggcttacaatcccttcaggatgcctacgacttccacggcttctcaaaacaccgtgtccaccacccctcggaggccgtcgactccacgcgccgcggtgacacaaacagcgtctcgggacgccgctgatgaggtttgggctttaagggacctttctggtggcggtagcaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggcccgtctggacgcagagcagcgagcgcagcaaaacggtacagattctttggacggacagactggcacgcattccgggggtggtagtctcacgagccagaaaccggtgctgggcaagcgagtcgcgacgccgcacgcgtccgcccgagcgcagacggtgacgtcgacgccggttcagggaaggctagagaaacaggtgtcgggcacgccgtcgacggtacccgccacgctgttgcaacctcaaccggcttcgtctaaaacgacgtcatcaaggaacgtgacttctggcgcgggaacctcttccgcttcttcggctcgacagccgtcagcctcggcgtccgttttgtcgcccacggaggatgatgtcgtgtcccccgccacatcgccgctgtccatgctttcgtcagcctctccgtccccggccaagagtgcccccccgtctccggtgaaaggccggggcagccgcgtcggtgttccttccttgaaacctactttgggcggcaaggcggtggtaggtcgaccgccctcggtccccgtgagcggtagcgcgccgggtcgcctgtccggcagctctggtggcggtagcggccgc

SEQ ID NO: 36 - protein, 412 aa, Artificial
  Synthetic Construct
TSRAGQPLVDITDTETSAKPPVTTAYKFEQPTLTFGAGVNVPAGAGAAILTPTPVNPSTAPAPAPTPTFAGTQTPVNGNSPWAPTAPLPGDMNPANWPRERAWALKNPHLAYNPFRMPTTSTASQNTVSTTPRRPSTPRAAVTQTASRDAADEVWALRDLSGGGSTKDTSLQAPPSYEESVYNSGRKGPGPPSSDASTAAPPYTNEQAYQMLLALARLDAEQRAQQNGTDSLDGQTGTHSGGGSLTSQKPVLGKRVATPHASARAQTVTSTPVQGRLEKQVSGTPSTVPATLLQPQPASSKTTSSRNVTSGAGTSSASSARQPSASASVLSPTEDDVVSPATSPLSMLSSASPSPAKSAPPSPVKGRGSRVGVPSLKPTLGGKAVVGRPPSVPVSGSAPGRLSGSSGGGSGR

SEQ ID NO: 37 - DNA, 33 bp, Artificial
  Synthetic
ggggatcccactagtcgtgctggccagccgctg

SEQ ID NO: 38 - DNA, 39 bp, Artificial
  Synthetic
ggtgctaccgccaccagaaaggtcccttaaagcccaaac

SEQ ID NO: 39 - DNA, 40 bp, Artificial
  Synthetic
cctttctggtggcggtagcaccaaagacacgtcgttacag

SEQ ID NO: 40 - DNA, 37 bp, Artificial
  Synthetic
gagactaccacccccggaatgcgtgccagtctgtccg

SEQ ID NO: 41 - DNA, 37 bp, Artificial
  Synthetic
cattccgggggtggtagtctcacgagccagaaaccgg

SEQ ID NO: 42 - DNA, 44 bp, Artificial
  Synthetic
ccagactcgagtcacccgcggccgctaccgccaccagagctgcc